(19) United States
(12) Patent Application Publication
(10) Pub. No.: US 2005/0004038 A1
(43) Pub. Date: Jan. 6, 2005

(54) Bivalent inhibitors of Glutathione-S-Transferases

(76) Inventors: Robert P. Lyon, Sammamish, WA (US); William M. Atkins, Seattle, WA (US); Dean Y. Maeda, Auburn, WA (US); John A. Zebala, Sammamish, WA (US)

Correspondence Address: JOHN ZEBALA, PRESIDENT;SYNTRIX BIOCHIP, INC, 215 CLAY STREET NW, SUITE B-5, AUBURN W, WA, 98001, US

(21) Appl. No.: 10/878,732

(22) Filed: Jun. 28, 2004

Related U.S. Application Data
(60) Provisional application No. 60/483,320, filed on Jun. 27, 2003.

(51) Int. Cl. A61K 38/08; A61K 38/06; C07K 7/06

(52) U.S. Cl. 514/17; 514/18; 530/329; 530/330; 530/331

(57) ABSTRACT

Bivalent inhibitors having affinity for one or more dimeric GST isozymes are provided. The bivalent inhibitors comprise two ligand domains connected by a molecular linker, wherein the ligand domains have affinity for one or more monomers in the one or more dimeric GST isozymes. The ligand domains are separated by a distance ranging from about 5 to about 100 Å. The bivalent inhibitors of the invention demonstrate greatly improved affinity for GST isozymes. In a specific embodiment, the bivalent inhibitors of the invention further provide affinity for substantially one GST isozyme and for substantially one GST class. The bivalent inhibitors of the invention have numerous uses that include the treatment of drug-resistant cancer, malaria, and stimulation of hematopoiesis.

The application claims the benefit of U.S. Provisional Patent application No. 60/483,320 filed Jun. 27, 2003, currently pending.

This invention was developed with government funding under National Institutes of Health Grant Nos. GM-62284GM, T32 GM07750 and R43CA92800. The U.S. Government may have certain rights in the invention.

### 1. FIELD OF THE INVENTION

The present invention relates to bivalent molecules having affinity for GSTs and to methods for their preparation and use. Such bivalent molecules are useful as inhibitors of GST enzyme activity and for blocking binding of GST by other proteins.

### 2. BACKGROUND OF THE INVENTION

Glutathione-S-Transferases (“GSTs”) are a large family of enzymes ubiquitously expressed in animals and plants that are involved in cellular defense against a broad spectrum of cytotoxic agents (see Gate and Tew, Expert Opin. Ther. Targets 5: 477, 2001). Over 400 different GST sequences have been identified and based on their genetic characteristics and substrate specificity can be classified in four different classes α, μ, π, and θ (see Mannervik et al., Biochem. J. 282:305, 1992). Each allelic variant encoded at the same gene locus is distinguished by a letter. In humans, there are currently five α class genes (GSTA1, GSTA2, GSTA3, GSTA4 and GSTω); five μ class genes (GSTM1, GSTM2, GSTM3, GSTM4 and GSTM5); four π class genes (GSTP1*A, GSTP1*B, GSTP1*C and GSTP1*D); and two θ class genes (GSTT1 and GSTT2). The various GST isozymes are dimeric proteins formed by the intra-class binary combinations of monomers. The monomeric proteins in a dimeric GST isozyme assume a fixed geometry with respect to one another (see Fritz-Wolf et al., Proc. Natl. Acad. Sci. 100: 13821, 2003).

These proteins are an extended family of Phase II xenobiotic metabolizing, (or ‘drug’ metabolizing) enzymes that catalyze the conjugation of glutathione (“GSH”) to a wide variety of exogenous electrophilic compounds including anticancer drugs, pesticides and genotoxic molecules. The first step in this process is the conversion of the GSH thiol group to thiolate which can, due to its high reactivity, attack the electrophilic nucleus of the substrate, leading to the formation of a glutathione-substrate conjugate. Glutathione conjugates of such compounds are generally more hydrophilic and less toxic and may be excreted more easily. GST contains two functional regions that participate in conjugation (see Dirr et al., Eur. J. Biochem. 220:645, 1994 and Armstrong, Chem. Res. Toxicol. 10:2, 1997). These include the N-terminal, hydrophilic domain that binds GSH (the G-site), and the C-terminal hydrophobic domain that binds the substrate (the H-site).

In yet further functions, GSTs possess glutathione peroxidase activity, with the GSTA1 isozyme providing the greatest specific activity (see Hurst et al., Biochem. J. 332:97, 1998). The glutathione peroxidase activity of GSTs protect cells against autocatalytic lipid peroxidation brought on by oxidative stress, and therefore plays a critical role in signaling and apoptosis triggered by lipid peroxidation products such as 4-hydroxynonenal (see Yang, et al., Acta Biochimica Polonica 50(2):319, 2003; Yang, et al., J. Biol. Chem. 276(22):19220, 2001; and Yang, et al., Invest. Ophthalmol. Vis. Sci. 43:434, 2002). GSTs thus perform a variety of beneficial physiologic functions that range from detoxification to regulation of cell signaling pathways.

GSTs also binds to other cellular proteins and regulates their activity. In particular, the GSTP1 isozyme regulates the stress response, cellular proliferation, and apoptosis by interacting with c-Jun NH2-terminal kinase (JNK) and so inhibiting its stress-induced kinase activity and promoting cell survival (see Ruscoe et al., J. Pharmacol. Exp. Ther. 298:339, 2001; Alder et al., EMBO J. 18:1321, 1999 and Wang et al., J. Biol. Chem. 276:20999, 2001). Accordingly, cancer cells that overexpress GST exhibit high JNK activity and escape apoptosis during exposure to anticancer drugs. Under stress, multimeric GST, which is normally inert, dissociates into monomers that are capable of activating JNK.

In the treatment of human pathology however, GSTs have been identified as playing a pivotal role in rendering a variety of therapeutic compounds ineffective. In particular, increased expression of GSTs in malignant neoplasms have been shown to be involved in their resistance to numerous anticancer agents (see Tew, Cancer Res. 54:4313, 1994). In infectious disease, increased expression of GST in strains of rodent and human malarial parasites correlates with resistance to chloroquine (see Srivastava et al., Trop. Med. Int. Health 4:251, 1999 and Harwaldt et al., Biol. Chem. 383:821, 2002). In a variety of disease states, the involvement of GST in cell signaling pathways can be used to therapeutically manipulate cell proliferation and apoptosis. Accordingly, potent inhibitors that neutralize GST catalysis and/or protein:protein binding would be desirable. Within this category, inhibitors that act specifically on particular GST classes or on specific isozymes would also be desirable.

Some monovalent GST inhibitors have been described (see Flatgaard et al., Cancer Chemother. Pharmacol. 33:63, 1993; Lyttle et al., J. Med. Chem. 37:189, 1994; and Kauvar and Lyttle, U.S. Pat. No. 5,763,570). These monovalent inhibitors potentiate the cytotoxic effect of numerous anticancer drugs in different cell lines and animal models (see Morgan et al., Cancer Chemother. Pharmacol. 37:363, 1996; and U.S. Pat. Nos. 5,763,570, 5,767,086 and 5,955,432). One of these inhibitors has also been shown to block the interaction between GSTP1-1 and JNK, activate downstream signaling pathways, and cause an early restoration in peripheral blood cells in an animal model of chemotherapy-induced myelosuppression (see U.S. Pat. Nos. 5,767,086 and 5,955,432). These results support the view that if effective, GST inhibition may be a therapeutic strategy for abrogating anticancer and antimalarial drug resistance, and stimulating hematopoiesis within a variety of clinical conditions.

These monovalent GST inhibitors, however, are ineffective: lacking sufficient potency they are incompatible with practical therapeutic development. To be effective for therapy, GST inhibitors must be sufficiently potent to develop them into therapeutics that can be produced cost-effectively and employed in daily dosages that are compatible with routine pharmaceutical formulations and patient compliance. In particular, these monovalent GST inhibitors fail because of their low affinity for GST isozymes: this necessitates huge daily dosages, which are incompatible with routine patient compliance and posing a formidable challenge for cost-effective pharmaceutical manufacturing.

Furthermore, effective GST inhibitors must be sufficiently selective for one or more GST isozymes to avoid a potential toxicity caused by inhibiting substantially all GST isozymes in a patient. However, known monovalent inhibitors demonstrate little selectivity in their affinity for GST isozymes since they inhibit multiple classes of GST isozymes. This potentially causes substantial side-effects and toxicity undercutting their therapeutic utility (see Petrini et al., Br. J. Haematol. 85:409, 1993). In general, finding compounds that specifically inhibit one isozyme over another is difficult because the isozymes all catalyze the same enzymatic reaction and have very similar active sites.

Small molecules have been covalently linked to produce bivalent inhibitors to increase affinities for acyl-CoA:cholesterol-O-acyltransferase and G protein coupled receptors (see U.S. Pat. Nos. 5,304,548 and 6,500,934, respectively), and corticotrophin receptors (Lin et al., Biochem. Pharmacol. 41(5):789, 1991). While this suggests that bivalency may increase the affinity of certain compounds, the targets were monomeric proteins and the sites bound by the inhibitors were free to vary, and whose orientation is fixed only once the bivalent inhibitor crosslinked two proteins. While bivalent inhibitors also increased affinities to thrombin (see U.S. Pat. Nos. 6,127,337 and 5,196,404) and hepatitis C virus NS3 protease (U.S. Pat. No. 5,990,276), these bivalent inhibitors are analogs of naturally occurring bivalent ligands (i.e. hirudin and the NS3-NS4A substrate) that bind to two different sites on the same monomeric protein.

### 3. SUMMARY OF THE INVENTION

Accordingly, there is a need in the art for a novel class of inhibitors which exhibit greater affinity and selectivity for one or more multimeric isozymes, and for dimeric GST isozymes in particular. The present invention fulfills this need and further provides other related advantages. One object of the invention is to provide bivalent inhibitors that bind to dimeric or multimeric proteins having monomeric constituents that assume a fixed geometry with respect to one another. Another object of the invention is to provide bivalent inhibitors capable of binding a site on each of two protein monomers attached to one another in a fixed geometry in a dimeric or multimeric protein, including those proteins for which there is no known bivalent ligand. Another object of the invention is to stabilize the quaternary structure of dimeric or multimeric proteins and inhibit their dissociation into monomers by binding a bivalent ligand to the dimeric or multimeric proteins. Another object of the invention is to provide bivalent inhibitors that increasing binding affinity by providing favorable interactions between GST dimers, and of promoting conversion of inhibitory monomers to non-inhibitory dimers.

One embodiment of the invention includes novel bivalent GST inhibitors that are useful as therapeutics for treating cancer; drug-resistant cancer; immunosuppression; immunosuppression during chemotherapy or radiotherapy; myelodysplasia; bone marrow transplantation; malaria; drug-resistant malaria; any of the disorders and conditions as disclosed in U.S. Pat. Nos. 5,763,570; 5,767,086; and 5,955,432, incorporated by reference, and related disorders involving one or more GST isozymes.

Another embodiment of the invention includes a bivalent inhibitor having affinity for a dimeric GST isozyme, wherein the bivalent inhibitor comprises two ligand domains covalently bonded to one another by a molecular linker, wherein the ligand domains have affinity for one or more monomers in the dimeric GST isozyme and are separated from one another by a distance ranging from about 5 to about 100 Å. In preferred embodiments, the molecular linker of the bivalent inhibitor enhances the affinity and/or increases isozyme selectivity.

Within certain embodiments, bivalent inhibitors of a dimeric GST isozyme are provided that comprise two ligand domains covalently bonded to one another by a molecular linker, wherein the bivalent inhibitors are represented by the formula:
D¹-L-D²
or a pharmaceutically acceptable salt thereof, where D¹ and D² are the two ligand domains and L is the molecular linker, wherein the ligand domains D¹ and D² are independently selected from the groups a) and b) consisting of:
a) amides; acids; or alkyl (1-10C), aryl (1-10C) or aralkyl (7-12C) esters of a monovalent radical of the formula: 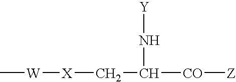
   wherein W is selected from the group consisting of a direct link or a mono- or disubstituted or unsubstituted hydrocarbon radical (1-20C) optionally containing 1 or 2 nonadjacent heteroatoms (O, S, or N), and wherein said substitution is selected from the group consisting of halo, —NO, —NO₂, —NR₂, —OR, and —SR, wherein R is H or lower alkyl (1-4C);
   wherein X is selected from the group consisting of S, O, CH₂ and direct link;
   wherein Y is selected from the group consisting of 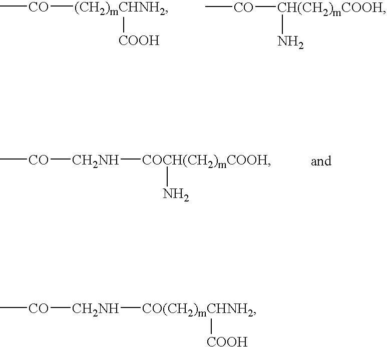
      wherein m is 1 or 2;
   wherein Z is selected from the group consisting of glycine, valine, alanine, β-alanine, 4-aminobutyric acid, aspartic acid, phenyl glycine, histidine, tryptophan, tyrosine, and phenylalanine, wherein the phenyl moiety of phenylalanine or phenyl glycine may optionally contain a single substitution selected from the group consisting of halo, OR, and SR, wherein R is H or alkyl (1-4C) linked through a peptide bond to the remainder of the compound;
b) amides; acids; or alkyl (1-10C), aryl (1-10C) or aralkyl (7-12C) esters of a monovalent radical selected from the group consisting of 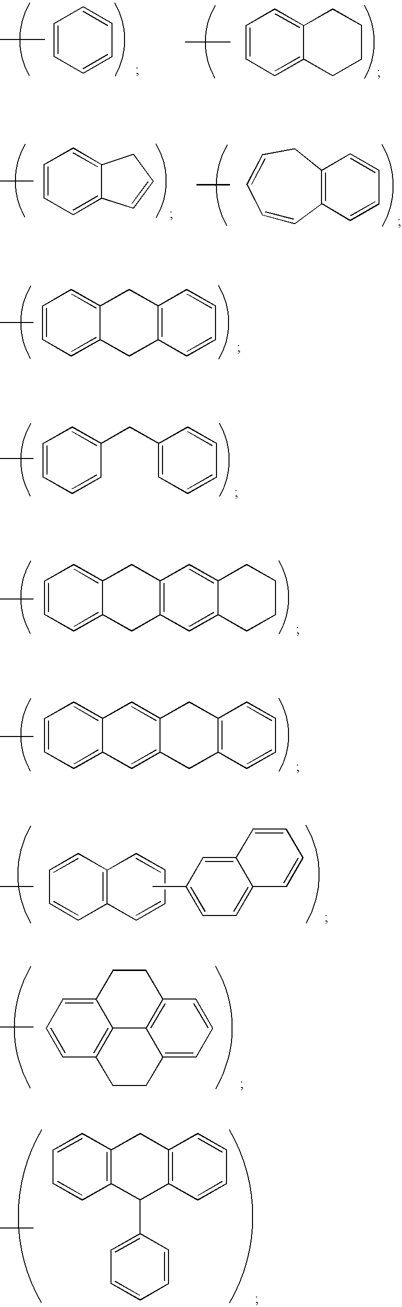 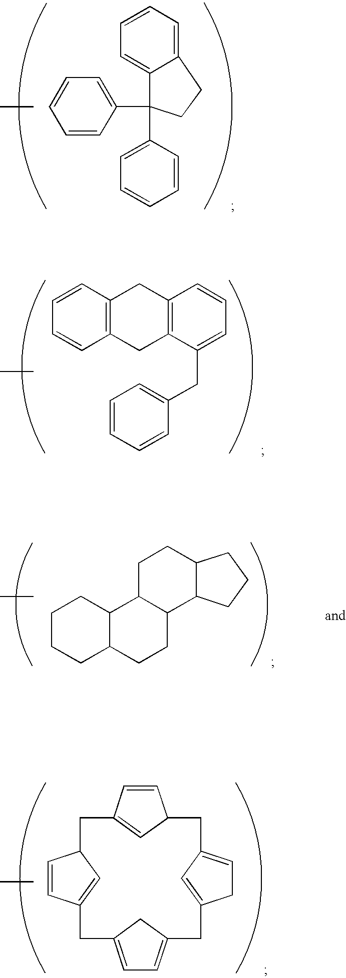
   wherein the monovalent radical may optionally be interrupted by at least one heteroatom selected from the group consisting of O, N and S;
   wherein the monovalent radical may optionally contain one or more substitutions selected from the group consisting of Cl, Br, F, I, —NH₂, —OH, ═O, —NO₂, —COOH, —CHO, —CO(alkyl), —CO(aryl), —SO₃H, —SO₂NH₂, —SO₂(alkyl), —SO₂(aryl), —CF₃, alkyl, alkoxyalkyl, aryl, and aralkyl;
the molecular linker L is selected from the group consisting of 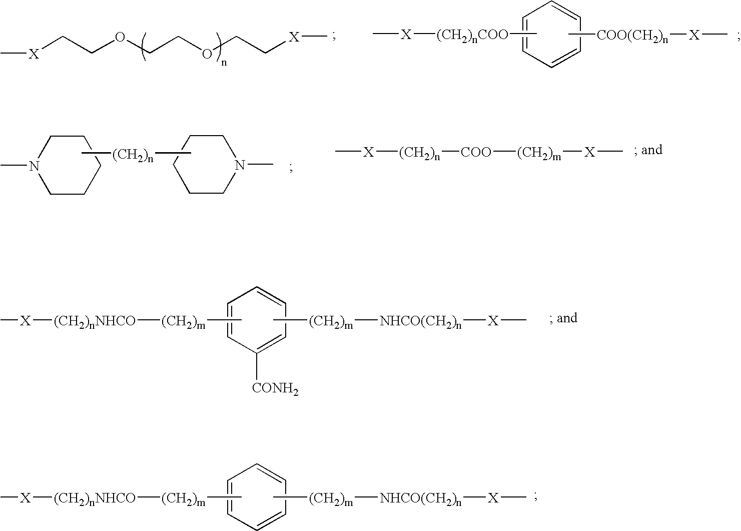
   wherein X is N, O, S, CH(AA)NH[COCH(AA)NH]ₖ, direct link, N-alkyl, N-aryl, CON-alkyl, or CO-alkyl; wherein AA is a natural or unnatural amino acid side chain;
   wherein k is 0-8, m is 0-8, n is 0-8; and
whereby the bivalent inhibitor to the dimeric GST isozyme has diverse properties.

Another embodiment of the invention is a bivalent inhibitor having affinity for a dimeric ST isozyme, wherein the bivalent inhibitor comprises two ligand domains covalently bonded to one another by a molecular linker, wherein the ligand domains are separated from one another by a distance ranging from about 5 to about 50 Å or 5 to about 25 Å. Another embodiment is a bivalent inhibitor, wherein the molecular linker separates the ligand domains with about 5 to about 100 molecular bonds. Another embodiment of the invention is a bivalent inhibitor, wherein the affinity between the bivalent inhibitor and the dimeric GST isozyme is characterized by a dissociation constant less than 1000 nM; less than 100 nM; less than 10 nM; or less than 1 nM.

Other embodiments of the invention provide a bivalent inhibitor with an affinity for a dimeric GST isozyme at least 10-fold, 100-fold, and more preferably 1000-fold greater than the affinity of each of the ligand domains for the dimeric GST isozyme. Another embodiment provides a bivalent inhibitor, wherein the molecular linker enhances the affinity of the bivalent inhibitor to the dimeric GST isozyme. Another embodiment provides a bivalent inhibitor, wherein the molecular linker increases isozyme selectivity of the bivalent inhibitor. Another embodiment provides a bivalent inhibitor, in which the affinity is for substantially one GST isozyme, preferably the GSTP1-1 isozyme. Another embodiment provides a bivalent inhibitor, in which the affinity is for substantially one GST class, preferably the π class of GST isozymes, and more preferably for substantially one GST isozyme. Another embodiment provides a bivalent inhibitor, in which the affinity for one GST class is 10-fold greater than the affinity for another GST class. Another embodiment provides a bivalent inhibitor, in which both ligand domains are the same structure. Another embodiment provides a bivalent inhibitor, in which the ligand domains are different structures. Another embodiment provides a bivalent inhibitor, in which the ligand domains are independently selected from the group consisting of glutathione, glutathione analogues, glutathione conjugates, ethacrynic acid, cibacron blue, uniblue A, doxorubicin, gossypol, hematin, rose bengal, sulfobromophthalein, indomethacin, piriprost, eosin b, eosin y, a synthetic or naturally occurring drug; a peptide, a small organic molecule; and mixtures thereof. Another embodiment provides a bivalent inhibitor, in which the molecular linker comprises a polynucleotide; a peptide; a saccharide; a cyclodextrin; a dextran; polyethylene glycol; polypropylene glycol; polyvinyl alcohol; a hydrocarbon; a polyacrylate; an alkyl chain interrupted by one or more atoms of O, S, or N atom, carbonyl, amide or aromatic group; an amino-, hydroxy-, thio- or carboxy-functionalized silicone; or a combination thereof.

Other embodiments provide a composition comprising the bivalent inhibitor in an amount effective for inhibiting a dimeric GST isozyme and a pharmaceutically acceptable carrier. In still other embodiments, the bivalent inhibitor is further derivatized with a toxin or chemotherapeutic drug. Other embodiments provide methods for synthesizing a bivalent inhibitor.

Another embodiment of the invention provides a method for inhibiting a dimeric GST isozyme in a cell, comprising preparing a bivalent inhibitor comprising two ligand domains covalently bonded to one another by a molecular linker, wherein the ligand domains have affinity for one or more monomers in the dimeric GST isozyme and are separated from one another by a distance ranging from about 5 to about 100 Å, and introducing the bivalent inhibitor to the cell. Another embodiment provides a method for inhibiting a dimeric GST isozyme in a cell, in which the cell comprises one GST isozyme, two GST isozymes, or more than two GST isozymes. Another embodiment provides a method for inhibiting a dimeric GST isozyme in a cell, comprising the additional step of removing the cell from a patient, and introducing the bivalent inhibitor to the cell outside of the patient, preferably a bone marrow cell.

Another embodiment of the invention provides a method for inhibiting a dimeric GST isozyme in a patient comprising preparing a bivalent inhibitor comprises two ligand domains covalently bonded to one another by a molecular linker, wherein the ligand domains have affinity for one or more monomers in the dimeric GST isozyme and are separated from one another by a distance ranging from about 5 to about 100 Å, and administering a therapeutically effective amount of a bivalent inhibitor or a pharmaceutically acceptable salt thereof to the patient. A preferred embodiment includes the additional step of derivatizing the molecular linker to optimize the absorption, distribution, metabolism, and excretion of the bivalent inhibitor within the patient. Another preferred embodiment provides treating a patient with a bivalent ligand, in which the molecular linker separates the ligand domains with about 5 to about 100 molecular bonds. Another preferred embodiment provides a method for treating a patient, in which the bivalent inhibitor is administered orally, transdermally or parenterally. Another preferred embodiment provides a method for treating a patient, in which the bivalent inhibitor is administered in an amount of from about 0.01 to about 10 mg/kg per dose. Another embodiment provides a method for treating a patient, in which substantially one GST isozyme is inhibited, preferably substantially one GST class is inhibited, and most preferably one GST isozyme is inhibited, particularly those from the π class of GST isozymes. Another embodiment provides a method for treating a patient, in which the bivalent inhibitor inhibits the enzyme catalysis of the dimeric GST isozyme, or inhibits binding the dimeric GST isozyme by another protein, or inhibits both enzyme catalysis and protein binding. Another embodiment provides a method for treating a patient, in which the GST isozyme inhibited is in a tumor, an infectious agent, or a bone marrow of the patient. Another embodiment provides a method for treating a patient, in which the toxin or chemotherapeutic agent is cleaved from the bivalent inhibitor after administering the derivatized bivalent inhibitor to the patient. Another embodiment provides a method for treating a patient, in which the linker is derivatized to optimize the absorption, distribution, metabolism or excretion of the bivalent inhibitor. Another embodiment provides a method for treating cancer; drug-resistant cancer; immunosuppression; immunosuppression during chemotherapy or radiotherapy; myelodysplasia; bone marrow transplantation; malaria; and drug-resistant malaria. Another embodiment provides a method for treating a patient, in which the bivalent inhibitor is used in combination with another pharmaceutical agent, preferably selected from the group consisting of melphan, doxorubicin, adriamycin, chlorambucil, cyclophosphamide, carboplatin, bleomycin, cisplatin, GM-CSF, G-CSF, cytokines, amifostine, and chloroquine.

Another embodiment of the invention provides a method for inhibiting substantially one multimeric isozyme, comprising preparing a bivalent inhibitor comprising two ligand domains covalently bonded to one another by a molecular linker, wherein the ligand domains have affinity for one or more monomers in the multimeric isozyme and are separated from one another by a distance substantially equivalent to the distance between the ligand domain binding sites on the multimeric isozyme, and contacting the multimeric isozyme with the bivalent inhibitor.

These and other aspects of the present invention will become apparent upon reference to the detailed description and illustrative examples which are intended to exemplify non-limiting embodiments of the invention. All references disclosed herein are hereby incorporated by reference in their entirety as if each was incorporated individually.

### 4. Glossary

Prior to setting forth the invention in detail, certain terms that will be used to describe aspects and embodiments of the invention are defined as follows:

“Affinity” means a binding interaction between a molecule and a protein, between two proteins, or between two molecules. Affinity can be measured and quantitated indirectly in an assay. Assays suitable for measuring affinity will be known to those skilled in the art, and for GSTs include, but are not limited to, a fluorescence resonance energy transfer (FRET) assay employing labeled GSTP1-1 and JNK (Wang et al., J. Biol. Chem. 276:20999, 2001), a chromogenic assay employing 1-chloro-2,4-dinitrobenzene (CDNB) (see Pickett and Lu, Ann. Rev. Biochem. 58:743, 1989), and in vivo assays employing chemotherapeutic potentiation and myelostimulation (see U.S. Pat. Nos. 5,767,086 and 5,955,432). Numerical values are often obtained from assays that are useful for quantitating affinity. These include, but are not limited to the dissociation constant (K_{d}), the 50% inhibitory concentration (IC₅₀), the Michaelis-Menten constant (Kₘ), and the inhibitory constant (Kₗ). As used in this disclosure, relative differences in such numerical values are used to express equivalent relative differences in affinity and vice versa. It will be understood that the determination of bivalent inhibitor affinity for one or more GST classes involves separately testing a representative GST isozyme from the one or more GST classes in an assay.

“Alkyl” means a saturated or unsaturated; unsubstituted or substituted (substituted by, for example, Cl, Br, F, I, —NH₂, —OH, ═O, —NO₂, —COOH, —SO₃H, —SO₂NH₂, —CF₃, or C₆₋₂₀ aryl); straight chain, branched chain, or cyclic hydrocarbon moiety having 1 to 20 carbon atoms and preferably from 1 to 6 carbon atoms this chain or cyclic hydrocarbon moiety may be interrupted by at least one heteroatom such as N, O or S.

“Aryl” means a carbocyclic moiety which may be substituted by, for example, Cl, Br, F, I, —NH₂, —OH, ═O, —NO₂, —COOH, —SO₃H, —SO₂NH₂, —SO₂(alkyl), —CF₃, or C₆₋₂₀ aryl; and containing one or more benzenoid-type rings preferably containing from 6 to 40 carbon atoms, this carbocyclic moiety may be interrupted by at least one heteroatom such as N, O or S.

“Aralkyl” means an aryl group attached to the adjacent atom by an alkyl group (e.g., benzyl), preferably containing from 6 to 30 carbon atoms.

“Alkoxyalkyl” means a substituted or unsubstituted alkyl group containing from 1 to 30 carbon atoms and preferably from 1 to 6 carbon atoms, wherein the alkyl group is covalently bonded to an adjacent element through an oxygen atom (e.g., methoxy and ethoxy).

“Enhances the affinity” means that a bivalent inhibitor has a molecular linker which endows it with 5-fold or greater affinity for one or more dimeric or multimeric isozymes than a bivalent inhibitor with equivalent spacing of the ligand domains but having a different molecular linker. Alternatively, “enhances the affinity” also means that a portion of a molecular linker attached to a single ligand domain (i.e. a “monovalent inhibitor”) has 5-fold or greater the affinity for one or more dimeric or multimeric isozymes than the single ligand domain alone. The portion attached must represent at least 50, 75, and most preferably greater than 90 weight percent of the molecular linker. As used herein, the portion attached is referred to as the “reference linker”.

“Fixed geometry” means that for other than thermal fluctuations in movement, monomers in a dimer or other multimer are in a fixed spatial position with respect to one another when the dimer or other multimer is otherwise unbound and not involved in the process of catalysis.

“Flexible” means that a linker comprises a plurality of rotatable bonds. A flexible molecular linker may be capable of assuming a variety of conformations.

“Increases isozyme selectivity” means that the ratio of affinities for a first and second isozyme for a bivalent inhibitor with a particular molecular linker is 5-fold or greater than a bivalent inhibitor with equivalent spacing of ligand domains, but having a different molecular linker. Alternatively, “increases isozyme selectivity” also means that a portion of a molecular linker attached to a single ligand domain (i.e. a “monovalent inhibitor”) preferentially binds to one of two isozymes in a ratio that is 5-fold or greater than the binding ratio of the ligand domain alone. The portion attached must represent at least 50, 75, and most preferably greater than 90 weight percent of the molecular linker. As used herein, the portion attached is referred to as the “reference linker”. It will be understood that in the case of increases in GST isozyme selectivity between isozyme classes, that the two GST isozymes are representative members from two isozyme classes.

“Inhibition” refers to contacting an isozyme with a molecule (i.e. an “inhibitor”) such that the molecule interferes with the ability of the isozyme (monomer or dimer) to engage in enzymatic catalysis, binding with another protein, or both. A molecule is said to cause “inhibition”, and thus “inhibit”, if the molecule can be contacted with the isozyme in a sufficient concentration to reduce enzymatic catalysis, binding with another protein, or both, by at least 50% as measured in an assay. In some embodiments, inhibition may be associated with covalent modification of the inhibitor although this is not a requirement of the invention. For example, GST may conjugate GSH to certain inhibitors, or itself become covalently attached to the inhibitor. In preferred embodiments, an inhibitor is not covalently modified during inhibition.

“Isozyme” refers to an enzyme that performs the same catalysis as another enzyme but is structurally different. Isozymes are also referred to as isoenzymes, and may be monomeric or multimeric (i.e. composed of two or more monomers or monomeric subunits). The difference in structure may be a difference in primary (i.e. amino acid sequence), secondary (e.g. alpha helical or beta sheet content), tertiary structure, and quaternary structure. For example, the spacing between active sites on two or more monomers in a multimeric enzyme may be different between two multimeric isozymes having the same catalytic activity (i.e. they have different quaternary structure). In a more specific example, dimeric protein tyrosine kinases all catalyze the hydrolysis of adenosine triphosphate (ATP), but the spacing between the catalytic sites for ATP differs between different protein tyrosine kinases (isozymes).

“Separated by a distance” or “separated from one another by a distance” means the distance separating the most terminal atoms of a molecular linker when the molecular linker is modeled in the most extended conformation.

“Substantially one GST class” means that a bivalent inhibitor or ligand domain has affinity for one GST isozyme belonging to a first isozyme class that is at least 2-fold, 5-fold, 10-fold, 100-fold, and more preferably 1000-fold greater than for at least one other GST isozyme belonging to an isozyme class different from the first.

“Substantially one GST isozyme” means that a bivalent inhibitor or ligand domain has affinity for a first GST isozyme that is at least 2-fold, 5-fold, 10-fold, 100-fold, and more preferably 1000-fold greater than for one or more other GST isozymes. It will be understood that the first GST isozyme and the one or more other GST isozymes may be from the same or different isozyme classes, or any combination thereof.

“Substantially one multimeric isozyme” means that a bivalent inhibitor or ligand domain has affinity for a first multimeric isozyme that is at least 2-fold, 5-fold, 10-fold, 100-fold, and more preferably 1000-fold greater than at least one other multimeric isozyme having the same catalytic activity as the first multimeric isozyme.

### 5. DETAILED DESCRIPTION OF THE INVENTION

### 5.1 Bivalent Inhibitors

The inventors have discovered that the present bivalent inhibitors exhibit potent inhibition of GSTs, of a specific GST isozyme class, and most preferably specific GST isozymes. In a preferred embodiment, bivalent inhibitors are provided that have affinity for one or more dimeric GST isozymes, wherein the bivalent inhibitors comprise two ligand domains connected by a molecular linker, and wherein each ligand domain has affinity for one or more GST monomers.

Within certain preferred embodiments, bivalent inhibitors of one or more dimeric GST isozymes are provided that comprise two ligand domains covalently bonded to one another by a molecular linker, wherein the bivalent inhibitors are represented by the formula:
D¹-L-D²
or a pharmaceutically acceptable salt thereof, where D¹ and D² are the two ligand domains and L is the molecular linker, wherein the ligand domains D¹ and D² are independently selected from the groups a) and b) consisting of:
a) amides; acids; or alkyl (1-10C), aryl (1-10C) or aralkyl (7-12C) esters of a monovalent radical of the formula: 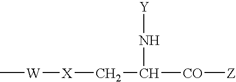
   wherein W is selected from the group consisting of a direct link or a mono- or disubstituted or unsubstituted hydrocarbon radical (1-20C) optionally containing 1 or 2 nonadjacent heteroatoms (O, S, or N), and wherein said substitution is selected from the group consisting of halo, —NO, —NO₂, —NR₂, —OR, and —SR, wherein R is H or lower alkyl (1-4C);
   wherein X is selected from the group consisting of S, O, CH₂ and direct link;
   wherein Y is selected from the group consisting of 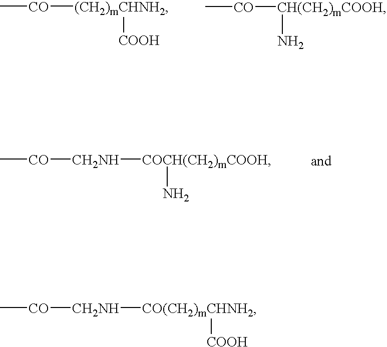
      wherein m is 1 or 2;
   wherein Z is selected from the group consisting of glycine, valine, alanine, β-alanine, 4-aminobutyric acid, aspartic acid, phenyl glycine, histidine, tryptophan, tyrosine, and phenylalanine, wherein the phenyl group of phenylalanine or phenyl glycine may optionally contain a single substitution selected from the group consisting of halo, OR, and SR, wherein R is H or alkyl (1-4C) linked through a peptide bond to the remainder of the compound;
b) amides; acids; or alkyl (1-10C), aryl (1-10C) or aralkyl (7-12C) esters of a monovalent radical selected from the group consisting of 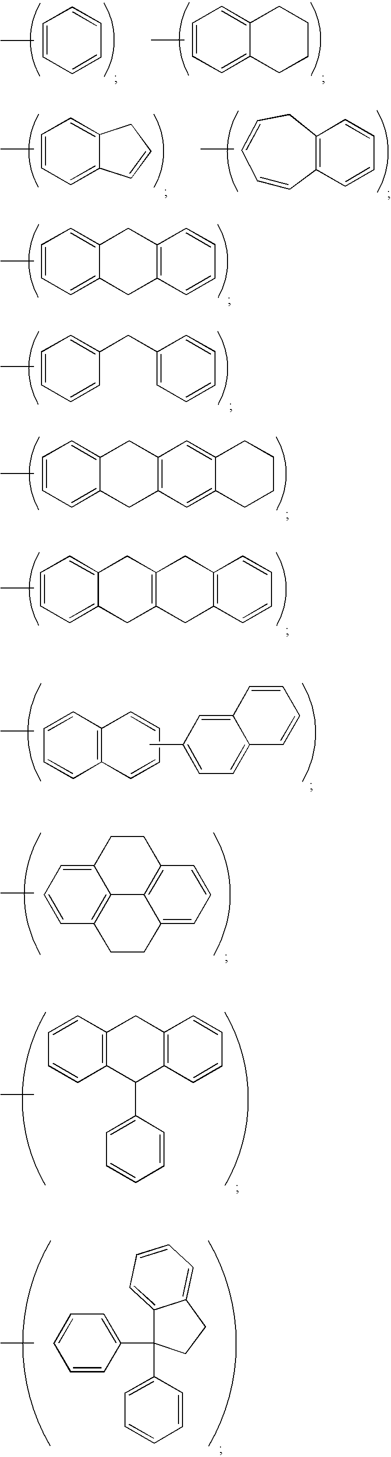 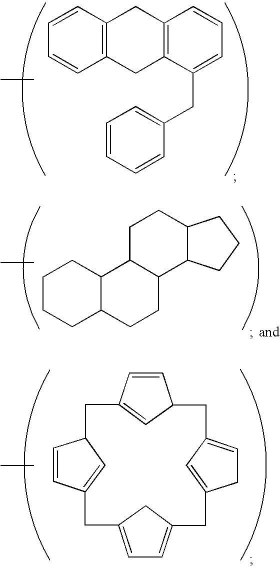
   wherein the monovalent radical may optionally be interrupted by at least one heteroatom selected from the group consisting of O, N and S;
   wherein the monovalent radical may optionally contain one or more substitutions selected from the group consisting of Cl, Br, F, I, —NH₂, —OH, ═O, —NO₂, —COOH, —CHO, —CO(alkyl), —CO(aryl), —SO₃H, —SO₂NH₂, —SO₂(alkyl), —SO₂(aryl), —CF₃, alkyl, alkoxyalkyl, aryl, and aralkyl;
the molecular linker L is selected from the group consisting of 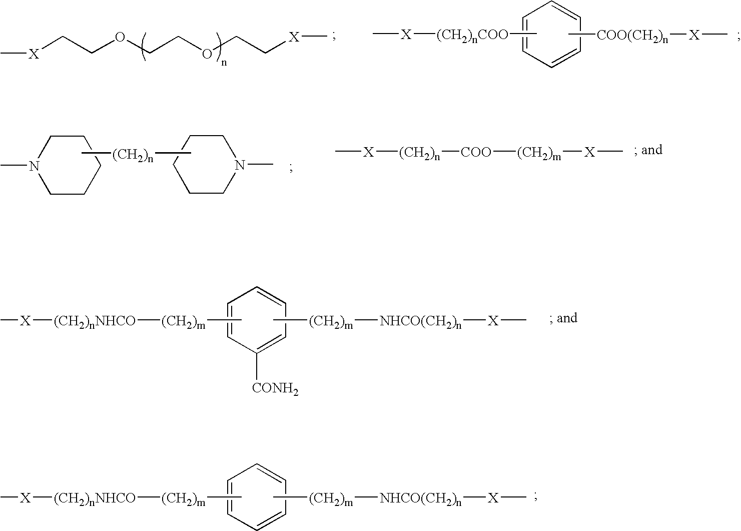
   wherein X is N, O, S, CH(AA)NH[COCH(AA)NH]ₖ, direct link, N-alkyl, N-aryl, CON-alkyl, or CO-alkyl; wherein AA is a natural or unnatural amino acid side chain;
   wherein k is 0-8, m is 0-8, n is 0-8; and
whereby said bivalent inhibitors to one or more dimeric GST isozymes have diverse properties.

In embodiments where the bivalent inhibitors comprise two connected ligand domains which by themselves have affinity for GSTs, the affinity of the bivalent inhibitor is higher than that of either of the two individual ligand domains. Thus, in preferred embodiments, the affinity between the bivalent inhibitor and a GST is at least 10-fold, 100-fold, and more preferably 1000-fold greater than the affinities of either of the individual ligand domains for the same GST. In absolute terms, the dissociation constant between the bivalent inhibitor and a GST is less than 1000 nM, less than 100 nM, less than 10 nM, and more preferably less than 1 nM. Accordingly, the bivalent inhibitors of the invention are effective GST inhibitors at markedly lower amounts than previously reported GST inhibitors. As such, the bivalent GST inhibitors of the invention should provide superior manufacturing economics, patient compliance, pharmaceutical formulations, and toxicity profiles.

The present invention provides bivalent inhibitors having affinity for GST dimers. Examples of GST dimers include, but are not limited to, those formed by the intra-class binary combination of human GST monomers comprising GSTA1, GSTA2, GSTA3, GSTA4, GSTω, GSTM1, GSTM2, GSTM3, GSTM4, GSTM5, GSTP1*A (also named GSTP1), GSTP1*B, GSTP1*C, GSTP1*D, GSTT1 and GSTT2. It is to be understood that monomers in a GST dimer are in a fixed geometry or orientation with respect to one another. In a preferred embodiment, the bivalent inhibitor has affinity for substantially one GST class, wherein the class is the π class or α class. In other preferred embodiments, the bivalent inhibitor has affinity for substantially one GST isozyme, wherein the GST isozyme is the GSTP1-1 or GSTA1-1 dimeric isozyme.

The bivalent inhibitors comprise two ligand domains that are molecular components each having affinity for one or more GST monomers and covalently coupled via a molecular linker. The ligand domains of the bivalent inhibitors of the present invention can be the same or different. The ligand domains facilitate binding between the bivalent inhibitors and one or more GST isozymes. Ligand domains may bind to any suitable location, or ‘binding site’, on the GST isozyme. Preferably, the binding site is a part of the active site on the GST isozyme such as the G or H site on each monomer, thus facilitating the use of active site analogues as ligand domains. In some embodiments, ligand domains having affinity for substantially one GST class or substantially one GST isozyme are useful, but not required, for preparing bivalent inhibitors with affinity for substantially one GST class or substantially one GST isozyme. In other preferred embodiments, the “one GST class” and “one GST isozyme” are the π class and the GSTP1-1 isozyme, respectively. In still other preferred embodiments, the “one GST class” and “one GST isozyme” are the α class and the GSTA1-1 isozyme, respectively.

According to the present invention, ligand domains are also compounds with affinity for one or more GSTs that when coupled to a molecular linker forms the bivalent inhibitor. Upon coupling, such compounds may lose atoms, acquire atoms, or undergo a molecular rearrangement. Thus, a compound is said to be a ligand domain if the portion coupled to the molecular linker comprises 50 to 150, 75 to 125, and more preferably 90 to 110 weight percent of the corresponding free compound.

Accordingly, useful ligand domains can be obtained by coupling to the molecular linker compounds (i.e. ligand domains) independently selected from the group consisting of glutathione, glutathione analogues, glutathione conjugates, ethacrynic acid, cibacron blue, uniblue A, doxorubicin, gossypol, hematin, rose bengal, sulfobromophthalein, indomethacin, piriprost, eosin b, eosin y, a synthetic or naturally occurring drug; a peptide, a small organic molecule, and any other moieties that have affinity toward GSTs; and mixtures thereof. Other useful compounds that can be coupled to the molecular linker to form ligand domains of the invention are disclosed in Mannervik et al., C.R.C. Crit. Rev. Biochem. 23:283, 1988; and U.S. Pat. No. 5,763,570.

In addition to the use of known GST inhibitors as ligand domains, potential compounds for use as ligand domains can be screened for by any methods known in the art, such as, for example, contacting a GST isozyme with the desired compound in an assay. If the GST is inhibited, the compound is an inhibitor. In other methods where a compound is not by itself a GST inhibitor, its suitability for use as a ligand domain can be ascertained by, for example, attaching the compound to a solid chromatography support, contacting the support with GST, and performing chromatography to ascertain affinity between the compound and a GST as disclosed in U.S. Pat. No. 5,763,570. Screening can be performed with GST isozymes selected from more than one isozyme class to identify those that have affinity for substantially one GST class. Other methods for screening compounds for their suitability as ligand domains will be apparent to those skilled in the art.

Ligand domains must have at a minimum, affinity for GSTs. More preferably ligand domains have an affinity characterized by a K_{d}, IC₅₀, Kₘ, or Kₗ whose numerical value is less than 1000 μM, 100 μM, 10 μM, and most preferably less than 1 μM. In preferred embodiments, ligand domains are by themselves inhibitors of GSTs, although this is not a requirement of the invention.

The ligand domains of the bivalent inhibitors are bonded covalently to a molecular linker that renders the ligand domains able to bind to the two monomers in a dimeric GST isozyme. A molecular linker consists of any biocompatible molecule functioning as a means to covalently link the two ligand domains. While the ligand domains can be situated anywhere on the molecular linker, each of the ligand domains is preferably located at each terminal end of the molecular linker. It will be understood that the particular composition of the molecular linker and attached ligand domains may often endow the bivalent inhibitor with rotational symmetry, although this in not a requirement of the invention and asymmetric bivalent inhibitors are also encompassed by the present invention.

Each ligand domain is linked to the molecular linker via a covalent bond. Suitable covalent bonds include, but are not limited to amide, ester, ether, thioether, amine, and methylene. Preferable bonds are those that are stable in vivo. Suitable linkers may range from flexible to rigid molecules. As use herein, rigid means that less than X % of bonds in the linker are rotatable. Preferred molecular linkers allow the two ligand domains bonded thereto to be separated by a distance ranging from about 5 to about 100 Å, 5 to about 75 Å, 5 to about 50 Å, and from about 5 to about 25 Å. In preferred embodiments, the linker comprises from about 5 to about 100 molecular bonds.

The spacing between the binding sites of the ligand domains varies in different dimeric GST isozymes, and this spacing can be used to select one or more molecular linkers useful for preparing a bivalent inhibitor with affinity for substantially one GST class or for substantially one GST isozyme. The spacing can be measured, or predicted theoretically, by any method known in the art. For example, molecular modeling can be used to determine spacing between binding sites for ligand domains in a particular GST isozyme, based upon, for example, the known x-ray crystallographic structure of a particular GST isozyme, or a particular GST isozyme co-crystallized with a ligand. Examples of GST isozymes that have been crystallized alone and/or with particular ligands include human pi (J. Molec. Biol., 227:214, 1992, and Structure 5:1287, 1997), human P1-1 (Biochem. 36:9690, 1997, and Biochem. 36:576, 1997), human mu (J. Molec. Biol. 338:815, 1994), human A1-1 (J. Molec. Biol. 232:192, 1993, and Structure 3:717, 1995), rat 3-3 (Biochem. 32:12949, 1993, and Biochem. 33:1043, 1994), rat mu (Biochem. 35:4753, 1996), mouse pi (J. Molec. Biol. 237:298, 1994), squid sigma (Biochem. 34:5317, 1995, and Proc. Natl. Acad. Sci. U.S.A. 93:8208, 1996), pig pi (J. Molec. Biol. 243:72, 1994), S. japonica (J. Molec. Biol. 255:289, 1996), and P. falciparum (Proc. Natl. Acad. Sci. U.S.A. 100(24):13821, 2003)

In another method of the invention, the appropriate spacing can be ascertained by forming a series of bivalent inhibitors wherein the ligand domains are separated by a distance that ranges from about 5 to about 100 Å, and then testing the affinity of the bivalent inhibitors for two or more GST isozymes in an assay. In preferred embodiments, at least two GST isozymes are tested that are selected from two or more isozyme classes.

It will be apparent to those of skill in the art that the use of spacing between the binding sites of the ligand domains may be exploited to control binding specificity to any multimeric isozyme besides dimeric GST isozymes. Thus, the spacing between the binding sites of the ligand domains varies in other multimeric isozymes, and this spacing can be used to select one or more molecular linkers useful for preparing a bivalent inhibitor for inhibiting substantially one multimeric isozyme. In particular, the length of a highly flexible linker may be used to selectively inhibit substantially one of a plurality of multimeric isozymes, wherein the multimeric isozyme inhibited has the shortest spacing between ligand binding sites of the plurality of multimeric isozymes. Alternatively, a more rigid linker may be used to selectively inhibit substantially one of a plurality of multimeric isozymes, wherein the spacing between ligand binding sites of the multimeric isozyme is either smaller or larger than the remainder of the plurality of multimeric isozymes.

As used herein, a bivalent inhibitor is considered to inhibit substantially one of a plurality of multimeric isozymes if its affinity is at least 5-fold, 10-fold, and more preferably greater than 50-fold greater than the affinity of any of the other multimeric isozymes. For example, cells contain a myriad of dimeric protein tyrosine kinases that all catalyze the hydrolysis of ATP during the phosphorylation of tyrosine. While kinase-specific inhibitors to the ATP active site are difficult to identify due to the similarity of the active sites, the spacing between ATP active sites of different dimeric protein tyrosine kinases is markedly different.

As previously noted for GST isozymes, the spacing can be measured, or predicted theoretically, by any method known in the art. For example, molecular modeling can be used to determine spacing between binding sites for ligand domains in a particular multimeric isozyme, based upon, for example, the known x-ray crystallographic structure of a particular multimeric isozyme. In another method, the appropriate spacing can be ascertained by forming a series of bivalent inhibitors wherein ligand domains are separated from one another by a distance substantially equivalent to the distance between at least two ligand domain binding sites on the multimeric isozyme, and then testing the affinity of the bivalent inhibitors for two or more multimeric isozymes in an assay. A ‘distance substantially equivalent’ means that the ligand domains will separated by a distance that ranges from about 50% to 150% of the distance between at least two ligand domain binding sites on the multimeric isozyme.

According to the present invention, molecular linkers are also compounds that when coupled to the ligand domains form the bivalent linker. Upon coupling, such a compound may lose atoms, acquire atoms, or undergo a molecular rearrangement. Thus, a compound is said to be a molecular linker if the portion coupled to the ligand domains comprises 50 to 150, 75 to 125, and more preferably 90 to 110 weight percent of the corresponding free compound.

Accordingly, suitable molecular linkers may comprise group(s) including, but not limited to, polynucleotides; peptides; saccharides; cyclodextrins and dextran; polymers including polyethylene glycol, polypropylene glycol, polyvinyl alcohol; hydrocarbons; polyacrylates; an alkyl chain interrupted by one or more atoms of O, S, or N atom, carbonyl, amide or aromatic group; amino-, hydroxy-, thio- or carboxy-functionalized silicones; and other biocompatible material units. Combinations of molecular linkers may also be used, comprising more than one class of the groups described above. For example, the molecular linker combinations can comprise saccharide and hydrocarbon groups; peptide, hydrocarbon and silicone groups; and the like; as well as being able to comprise more than one member within a class. Where the molecular linker comprises more than one class of a group, it is preferably obtained by joining different units via their functional groups. For example, in the case of a molecular linker comprising a saccharide and a nucleophilic group-bearing unit such as polyethylene glycol, polypropylene glycol, polyvinyl alcohol, silicone, or a peptide comprising serine or tyrosine, the hydroxyl group-bearing groups can form stable glycosidic bonds with the glycoside terminus of a saccharide. Methods for forming such bonds involve standard organic synthesis and are well known to those of ordinary skill in the art.

Other suitable molecular linkers of the invention are disclosed in U.S. Pat. Nos. 5,304,548 and 6,127,337; Desk Reference of Functional Polymers: Synthesis and Applications, Arshady, R. (ed.) (1997), American Chemical Society, Washington, D.C.; and the references disclosed therein. Such molecular linker materials described above are widely commercially available or obtainable via synthetic organic methods commonly known to those skilled in the art.

In preferred embodiments wherein the bivalent inhibitor is used as an in vivo therapeutic, the composition of the molecular linker will be varied to optimize the absorption, distribution, metabolism and excretion of the bivalent inhibitor (i.e. the “ADME” properties of the bivalent inhibitor). In various embodiments, the molecular linker is modified so as to increase or decrease the bivalent inhibitor's hydrophobicity, number of H-bond donors and acceptors, and the number of rotatable bonds (see Veber et al., J. Med. Chem. 6:2615, 2002). In other embodiments the molecular linker is modified to facilitate cellular uptake by, for example, covalently attaching folic acid to the molecular linker so the bivalent inhibitor is recognized by the folate receptor (see Low et al., 8^{th} International Symposium on Recent Advances in Drug Delivery Systems (1997), Salt Lake City, Utah, pp. 48-50 and Leamon et al., J. Biol. Chem. 268:24847, 1992). Optimizing the ADME properties employs testing bivalent inhibitors with different molecular linker compositions using methods well known to those skilled in the art. These methods include, but are not limited to, solubility testing; partitioning in biphasic mixtures of n-octanol and water; transport across Caco-2 and MDCK cell lines; binding to plasma proteins; degradation in plasma and whole blood; degradation in liver microsomes; degradation in isolated fractions of the P450 system; and monitoring and characterizing the absorption, distribution, metabolism and excretion of isotopically-labeled and unlabeled bivalent inhibitors in rodent, canine, primate, and human species.

The bivalent inhibitor can optionally comprise nucleophilic or electrophilic functional groups which can form covalent bonds with another pharmaceutical species such as a toxin or chemotherapeutic agent, so as to provide a means to target this pharmaceutical species to cells, tissues and tumors that produce particular GST isozymes. Examples of suitable nucleophilic groups include amino, hydroxyl and sulfhydryl groups. Examples of suitable electrophilic groups include carboxyl, epoxide, ester, acid halide groups and their equivalents. Where the pharmaceutical species forms covalent bonds with the functional groups of the bivalent inhibitor, the inhibitor can comprise amino, hydroxyl or sulfhydryl groups which can form amide, ester or thioester bonds, respectively, with a carboxyl group, or its equivalent, of the pharmaceutical species, and vice versa. Similarly, epoxide-functionalized bivalent inhibitors can form stable adducts with free amino, hydroxyl or sulfhydryl groups of the pharmaceutical species, and vice versa.

In one embodiment the pharmaceutical species is coupled to the molecular linker. In another embodiment the pharmaceutical species is coupled to one or both of the ligand domains. In a preferred embodiment, the pharmaceutical species coupled to the bivalent inhibitor can be cleaved from the bivalent inhibitor after administration to a patient, releasing the free pharmaceutical species. Covalent bonds suitable for in vivo cleavage of the pharmaceutical species will be well known to those skilled in the art. These include, but are not limited to, esters cleavable by non-specific intracellular esterases, GST-catalyzed cleavage of sulfones (see Gate and Tew, Expert Opin. Ther. Targets 5: 477, 2001), and GST-catalyzed cleavage of sulfonamides (see Zhao et al., Drug Metab. Dispos. 27:992, 1999).

In an alternative embodiment, the bivalent inhibitors of the present invention may be labeled with one or more radioisotopes. The choice of radioisotope is based upon a number of well-known factors, for example, toxicity, biological half-life and detectability. Preferred radioisotopes include, but are not limited to ¹²⁵I, ¹²³I and ¹¹¹I. Techniques for labeling the compounds of this invention are well known in the art. Most preferably, the radioisotope is ¹²³I and the labeling is achieved using ¹²³I-Bolton-Hunter Reagent. The labeled bivalent GST inhibitor is administered to a patient and allowed to bind to one or more GST isozymes. The bound regions are then observed by utilizing well-known detecting means, such as a camera capable of detecting radioactivity coupled to a computer imaging system. Radiolabeled bivalent GST inhibitors will have use in diagnostic imaging of the distribution of GST isozymes in living patients. As noted by the prior art, it is now well established that GSTs, especially the GST π class, are overexpressed with high frequency in a wide variety of tumors including breast, colon, esophagus, kidney and lung, whereas GST levels in normal surrounding tissues are low (see Gate and Tew, Expert Opin. Ther. Targets 5: 477, 2001).

In another embodiment, the bivalent inhibitors of the present invention may be further derivatized and used as analytical reagents and in chromatography as disclosed in U.S. Pat. No. 5,763,570, and references cited therein. In such embodiments, the derivatized or underivatized bivalent inhibitor can be used to capture a GST or GST-labeled fusion protein on a solid-support such as a chromatography support or on a microtiter dish. In some embodiments, the bivalent inhibitors may be derivatized with a label, such as, for example, a fluorophore or quantum dot. Labeled bivalent inhibitors will find used as detection agents able to detect the presence of a GST isozyme or GST fusions proteins in cellular extracts or within cells themselves.

In particular embodiments, the molecular linker may or may not interact with the GST isozyme. In embodiments where the molecular linker interacts with the GST isozyme, the molecular linker can comprise a particular functional group composition that enhances the affinity of the bivalent inhibitor for one or more dimeric GST isozymes by binding to exposed functional groups on the surface of the isozymes. The functional groups on the molecular linker and on the surface of the GST can include, but are not limited to, nucleophiles, electrophiles, hydrogen-bond donors, hydrogen-bond acceptors, thiols, ethers, thioethers, amide bonds, esters, carbonyls, ketones, aryl, aromatic groups, any natural and un-natural amino acid side-chain, and charged functional groups, such as for example, ammonium groups or carboxylate groups.

Potential molecular linkers that enhance the affinity of a bivalent inhibitor can be screened for by any methods known in the art. In one representative method, monovalent inhibitors are prepared, wherein a reference linker is employed that comprises a particular functional group composition, but which is coupled with only one ligand domain. If the affinity of the monovalent inhibitor has greater than 5-fold the affinity for one or more dimeric GST isozymes than the ligand domain alone (i.e. a separate compound, as defined above), it is said that a molecular linker comprising at least 50, 75, and most preferably greater than 90 weight percent of the reference linker enhances the affinity of a bivalent inhibitor.

In another representative method, potential molecular linkers that enhance the affinity of a bivalent inhibitor can be screened for by contacting a GST isozyme with a library of bivalent inhibitors wherein the ligand domains and their spacing are the same in each library member but the composition of the molecular linker is varied. Methods for creating such libraries are well known in the art. By way of example, library members can be screened either free in-solution or attached to solid-supports such as beads (e.g. PEG-PS, Novabiochem, Inc.) or a flat-glass surface (i.e. a “chip array”). The preparation of such chip arrays is described in co-pending application entitled, “Method and Composition For Performing an Array of Chemical Reactions on a Support Surface” (U.S. patent application Ser. No. 09/326,479).

The affinities of each library member for the GST isozyme are then determined. The in-solution affinities of the library members for GST can be detected using an assay (e.g. CDNB, FRET). Assays suitable for detecting affinity on a solid-support include, for example, contacting a support-bound library with fluorescently-labeled GST, or by contacting a support-bound library with GST and then performing chromatography as disclosed in U.S. Pat. No. 5,763,570. If the relative affinities of at least two library members differs by at least 5-fold, the bivalent inhibitor with the greater affinity is said to have a molecular linker that enhances the affinity of the bivalent inhibitor. Other methods for identifying molecular linkers that enhance the affinity of bivalent inhibitors for one or more GST isozymes will be familiar to those in the art.

Within the invention, it will be understood that a molecular linker that enhances the affinity of a bivalent inhibitor may result in isozyme selectivity that is decreased, unchanged, or increased. Preferably, the molecular linker both enhances the affinity and increases isozyme selectivity, such that the bivalent inhibitor has affinity for substantially one GST class, and more preferably for substantially one GST isozyme.

A potential molecular linker that increases isozyme selectivity of a bivalent inhibitor can be screened for by any methods known in the art. In one representative method, the monovalent inhibitor (i.e. with attached reference linker) and ligand domain described above are separately contacted with a first and second GST isozyme, and their affinity for each of the GST isozymes ascertained. The affinities of the monovalent inhibitor for the first and second GST isozymes are expressed as a first ratio (i.e. the ratio of the first to the second GST isozyme). The affinities of the ligand domain for the first and second GST isozymes are expressed as a second ratio (i.e. the ratio of the first to the second GST isozyme). If the first ratio is 5-fold or greater than the second ratio, then the reference linker “increases isozyme selectivity” for the first GST isozyme. Conversely, if the inverse of the first ratio is 5-fold or greater than the inverse of the second ratio, then the reference linker “increases isozyme selectivity” for the second GST isozyme. The molecular linker of a bivalent inhibitor “increases isozyme selectivity” for the same GST isozyme as the reference linker if it contains at least 50, 75, and most preferably greater than 90 weight percent of the reference linker.

In another screening method, a first and second GST isozyme are separately contacted with the support-bound libraries described above (i.e. each library member having equivalent ligand domain spacing), and for each library member the ratio of the first to the second GST isozyme affinity is ascertained. If the ratios of at least two library members differ from one another by 5-fold or greater, the bivalent inhibitor with the greater ratio is said to have a molecular linker that “increases isozyme selectivity” for the first GST isozyme, and the bivalent inhibitor with the smaller ratio is said to have a molecular linker that “increases isozyme selectivity” for the second GST isozyme. Other methods for identifying a molecular linker that increases isozyme selectivity of bivalent inhibitors will be familiar to those in the art.

It will be apparent to those skilled in the art that in such screening methods, two or more GST isozymes could be employed simultaneously to determine affinities to ligand domains, monovalent inhibitors, and bivalent inhibitors, using for example, GST isozymes differentially labeled with fluorophores having non-overlapping emission spectra. It is also within the purview of one of skill in the art to select a particular molecular linker composition that optimizes ADME, enhances the affinity, and increases isozyme selectivity of the bivalent inhibitors of the present invention. It should be noted that a person skilled in the art could substitute suitable molecular linkers and synthesize variants of such active bivalent inhibitors as disclosed herein.

### 5.2 Methods and Synthons Useful for Synthesizing Bivalent GST Inhibitors

The bivalent GST inhibitors of the present invention can be synthesized by a suitable strategy such as by classical solution synthesis, exclusively solid-phase synthesis, or a combination of both strategies. In general, bivalent GST inhibitors can be synthesized by the condensation of synthons. Synthons are chemical building blocks that contribute to the molecular linker or ligand domains of the bivalent inhibitor. Synthons bear one or more reactive moieties that include, but are not limited to, carboxyl, acid chloride, hydroxyl, amino, thiol, alkylene, aryl halide, and alkyl halide. Synthons are condensed in-solution or on the solid-phase after modification by activation, by protection with suitable protecting groups, or a combination thereof. In some embodiments, synthons are condensed in-solution or on the solid-phase without modification.

Synthons suitable for assembly of the molecular linker may be obtained from the group that includes, but is not limited to, natural amino acids, unnatural amino acids, diols, diamines, haloalkyl acids, polyoxyethylene derivatives, and those compounds disclosed in Gammill et al., U.S. Pat. No. 5,304,548; Konishi et al., U.S. Pat. No. 6,127,337; Desk Reference of Functional Polymers: Synthesis and Applications, edited by Reza Arshady, (1997), American Chemical Society, Washington, D.C.; and the references disclosed therein. Other compounds useful for providing synthons for assembly of the molecular linker include; 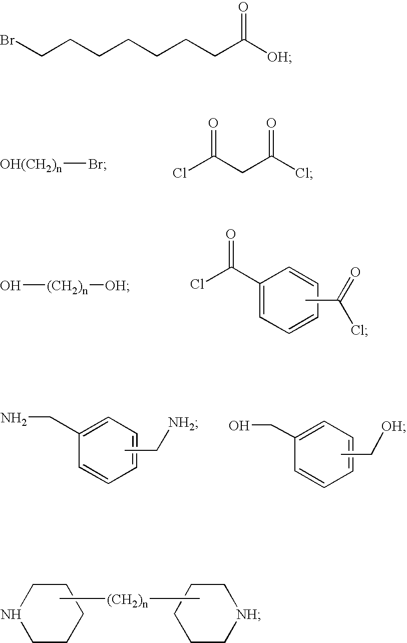
OH—CH₂CH₂—OH; OH—CH₂CH₂CH₂—OH; H—(OCH₂CH₂)ₙ—OH; NH₂—(CH₂)ₙ—NH₂; NH₂—CH₂CH₂—(OCH₂CH₂)ₙ—NH₂; NH₂—(CH₂)ₙ—COOH; and Br—CH₂—COOH; where n=1 to about 20.

Synthons suitable for assembly of the ligand domains may be obtained from the group that includes, but is not limited to, natural amino acids, unnatural amino acids, glutathione, glutathione analogues, glutathione conjugates, ethacrynic acid, cibacron blue, uniblue A, doxorubicin, gossypol, hematin, rose bengal, sulfobromophthalein, indomethacin, piriprost, eosin b, eosin y, and compounds as disclosed in Mannervik et al., C.R.C. Crit. Rev. Biochem. 23:283, 1988; and Kauvar and Lyttle, U.S. Pat. No. 5,763,570. Other compounds useful for providing synthons for assembly of the ligand domains include:

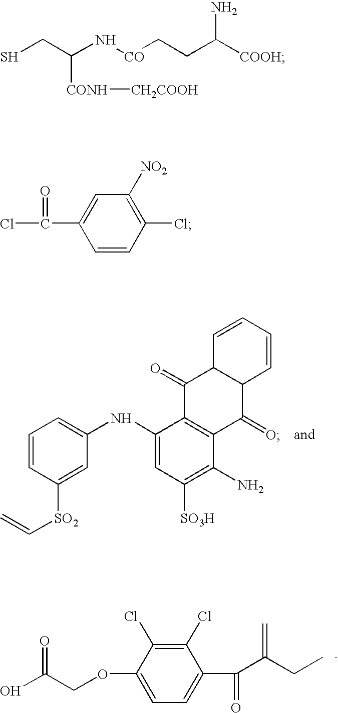

According to one embodiment of an in-solution synthesis strategy, the bivalent GST inhibitors of the present invention are prepared by condensing 8-bromooctanoic acid with a variety of ω-bromo alkyl alcohols to yield asymmetric molecular linkers. The molecular linkers are further derivatized by displacing the reactive bromine on these linkers with the thiol of glutathione to produce bis-glutathionyl bivalent inhibitors.

In another embodiment of an in-solution strategy, isophthaloyl chloride is condensed with diols to yield symmetric molecular linkers bearing reactive hydroxyls. These molecular linkers and a variety of polyethylene glycols are esterified to 4-chloro-3-nitro-benzoyl chloride which is further coupled with glutathione. The resultant bivalent inhibitors comprise ligand domains of 4-(S-glutathione)-3-nitro-benzoyl conjugates.

In another embodiment of an in-solution strategy, isophthaloyl chloride is condensed with a variety of diamines to yield symmetric molecular linkers bearing reactive amines. These molecular linkers and a variety of bis-amino-(polyoxyethylene) derivatives are condensed with uniblue A to provide symmetric bivalent inhibitors bearing uniblue A as the ligand domains.

According to an embodiment of a solid-phase synthesis strategy, the bivalent GST inhibitors of the present invention are prepared by the bi-directional assembly of suitably protected synthons off a resin-bound bifunctional synthon (see Merrifield, J. Am. Chem. Soc. 85:2149, 1963). Where synthons bear two or more reactive moieties, all but one reactive moiety are protected with suitable protecting groups to prevent undesired chemical reactions from occurring during the assembly of synthons off the bifunctional synthon. After two synthons are attached per bifunctional synthon, one additional protecting group is selectively removed from each of the synthons to allow subsequent couplings with other synthons. The conditions for the removal of this protecting group do not remove the other protecting groups that may exist elsewhere in the evolving resin-bound structure.

In some embodiments, selective removal of a protecting group from the most recently coupled synthon is not required to couple a subsequent synthon as, for example, is the case for the bromoacetic acid synthon. The process of coupling synthons is repeated until the last synthon is coupled. The last synthon will, in some embodiments, not have any protecting groups.

Suitable protecting groups are those known to be useful in the art of stepwise solid-phase synthesis. Included are acyl type protecting groups (e.g., formyl, trifluoroacetyl, acetyl), aryl type protecting groups (e.g., biotinyl), aromatic urethane type protecting groups (e.g., benzyloxycarbonyl (Cbz), substituted benzyloxycarbonyl and 9-fluorenylmethyloxy-carbonyl (Fmoc)), aliphatic urethane protecting groups (e.g., t-butyloxycarbonyl (BOC), isopropyloxycarbonyl, cyclohexyloxycarbonyl) and alkyl type protecting groups (e.g., benzyl, triphenylmethyl). Other protecting groups will be known to those skilled in the art and include, for example, those disclosed in T. W. Greene and P. G. M. Wuts, Protective Groups in Organic Synthesis, 3^{rd} Edition, John Wiley & Sons, New York, 1999. In certain embodiments, protecting groups are phthaloyl or Fmoc, thus the bivalent inhibitors are said to be synthesized by phthaloyl or Fmoc chemistry, respectively.

It will be apparent to those of skill in the art that the assembly of synthons off a bifunctional synthon can proceed in a symmetric or asymmetric fashion. In symmetric assembly, the resin-bound bifunctional synthon is protected on both reactive ends with the same protecting group. Protecting group removal exposes both reactive ends to synthon coupling and assembly occurs symmetrically. In asymmetric assembly, the resin-bound bifunctional synthon is orthogonally protected on its reactive ends such that each protecting group can be removed selectively. Selective removal allows synthon coupling and assembly to occur on one reactive end only. Depending on when during assembly the second protecting group is removed, the assembly of synthons may be totally asymmetrical or partially asymmetrical. A compound suitable for forming a resin-bound bifunctional synthon according to the present invention is diaminomethylbenzoic acid (DAMBA):

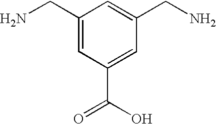

The synthon protecting groups not participating in the assembly process must remain intact during coupling (i.e. the protecting groups on side-chains). However, such protecting groups must be removable upon the completion of synthesis, using reaction conditions that will not alter the finished bivalent inhibitor. In Fmoc chemistry, such protecting groups are mostly t-butyl or trityl based. In Fmoc chemistry, the preferred protecting groups are 2,2,5,7,8-pentamethylchroman-6-sulfonyl (Pmc) or 2,2,4,6,7-pentamethyldihydrobenzofuran-5-sulfonyl (Pbf) for Arg; trityl for Asn, Cys, Gln and His; tert-butyl for Asp, Glu, Ser, Thr and Tyr; and tBoc for Lys and Trp.

Solid phase synthesis is usually carried out from a carboxyl-terminus by coupling the amino protected bifunctional synthon to a suitable solid support. An ester linkage is formed when the attachment is made to a chloromethyl, chlorotrityl or hydroxymethyl resin, and the resulting bivalent inhibitor will have a free carboxyl group on the molecular linker. Alternatively, when an amide resin such as benzhydrylamine, Rink amide or PAL resin is used, an amide bond is formed and the resulting bivalent inhibitor will have a free carboxamide group on the molecular linker. These resins, whether polystyrene- or polyamide-based or polyethylene glycol-grafted, are commercially available, and their preparations have been described (see Stewart et al., Solid Phase Peptide Synthesis, 2^{nd} Edition, Pierce Chemical Co., Rockford, Ill., 1984; Bayer & Rapp, Chem. Pept. Prot. 3:3, 1986; and Atherton, et al. Solid Phase Peptide Synthesis: A Practical Approach, IRL Press, Oxford, 1989).

The protected bifunctional synthon is attached to a hydroxyethyl resin using various activating agents including dicyclohexylcarbodiimide (DCC), N,N′-diisopropylcarbodiimide (DIPCDI) and carbonyldiimidazole (CDI). It can be attached to chloromethyl or chlorotrityl resin directly in its cesium tetramethylammonium salt form or in the presence of triethylamine (TEA) or diisopropylethylamine (DIPEA). Attachment to an amide resin employs the same method as for amide bond formation during synthon coupling reactions.

Following the attachment of the bifunctional synthon to the resin support, the amino protecting groups are removed using various reagents depending on the protecting chemistry (e.g. hydrazine for phthaloyl, or piperidine for Fmoc). The extent of Fmoc removal can be monitored at 300-320 nm. After removal of the amino protecting group, the remaining protected synthons are coupled stepwise in the required order to obtain the desired resin-bound bivalent inhibitor.

Various activating agents can be used for the coupling reactions including DCC, DIPCDI, 2-chloro-1,3-dimethylimidium hexafluorophosphate (CIP), benzotriazol-1-yl-oxy-tris-(dimethylamino)-phosphonium hexafluorophosphate (BOP) and its pyrrolidine analog (PyBOP), bromo-tris-pyrrolidino-phosphonium hexafluorophosphate (PyBroP), N-[(1H-benzotriazol-1-yl)-(dimethylamino)methylene]-N-methylmethanaminium hexaflourophosphate N-oxide (HBTU) and its tetrafluoroborate analog (TBTU) or its pyrrolidine analog (HBPyU), (HATU) and its tetrafluoroborate analog (TATU) or pyrrolidine analog (HAPyU). The most common catalytic additives used in coupling reactions include 4-dimethylaminopyridine (DMAP), 3-hydroxy-3,4-dihydro-4-oxo-1,2,3-benzotriazine (HODhbt), N-hydroxybenzotriazole (HOBt) and 1-hydroxy-7-azabenzotriazole (HOAt). Synthon acid fluorides or chlorides may be used for difficult couplings. Each protected synthon is used in excess (>2.0 equivalents), and the couplings are usually carried out in NMP (“N-methylpyrrolidone”) or in DMF (“N,N-dimethylformamide”), CH₂Cl₂ or mixtures thereof. The extent of completion of the coupling reaction can be monitored at each stage, e.g., by the ninhydrin reaction (see Kaiser et al., Anal. Biochem. 34:595, 1970). In cases where incomplete coupling is found, the coupling reaction is extended and repeated and may have chaotropic salts added. The coupling reactions can be performed automatically with commercially available instruments.

After the entire assembly of the desired bivalent inhibitor, the resin-bound bivalent inhibitor is cleaved with a reagent with proper scavengers. The Fmoc bivalent inhibitor are usually cleaved and deprotected by TFA (“trifluoroacetic acid”) with scavengers (e.g., H₂O, ethanedithiol, phenol and thioanisole).

### 5.3 Compositions Comprising Bivalent Inhibitors and Methods of Treatment Employing such Compositions

While it may be possible that, for use in therapy, a bivalent GST inhibitor of the invention may be administered as the purified chemical, it is preferable to present the active ingredient as a pharmaceutical formulation.

It will be appreciated by those skilled in the art that the bivalent GST inhibitors may be modified to provide pharmaceutically acceptable salts thereof which are included within the scope of the invention. Pharmaceutically acceptable salts of the bivalent inhibitors include those derived from pharmaceutically acceptable inorganic and organic acids and bases. Examples of suitable acids include hydrochloric, hydrobromic, sulphuric, nitric, perchloric, fumaric, maleic, phosphoric, glycollic, lactic, salicylic, succinic, toluene-p-sulphonic, tartaric, acetic, citric, methanesulphonic, formic, benzoic, malonic, naphthalene-2-sulphonic and benzenesulphonic acids. Other acids such as oxalic, while not pharmaceutically acceptable, may be useful as intermediates in obtaining the bivalent GST inhibitors of the invention and their pharmaceutically acceptable acid addition salts.

It will also be appreciated by those skilled in the art that the bivalent GST inhibitors may be modified to provide pharmaceutically acceptable prodrug forms which are included within the scope of the invention, such as for example, the esterified forms of a bivalent GST inhibitor. As therapeutic agents, the esterified bivalent GST inhibitors of the invention may be useful to facilitate permeation of the cell membrane. Particularly preferred esters are the ethyl esters, benzyl esters and derivatized benzyl esters. The esterified forms offer the opportunity to modify the invention compounds in ways that affect their suitability as in vivo and ex vivo therapeutic agents, as well as diagnostic reagents. The esters may contain additional useful ligands such as hydrophobic moieties to enhance cell permeation, and reactive groups to facilitate linkage to other substances.

The invention thus further provides a pharmaceutical formulation comprising a bivalent GST inhibitor in a pharmaceutically acceptable addition salt or prodrug form thereof together with one or more pharmaceutically acceptable carriers therefor and, optionally, other therapeutic ingredients. The carrier(s) must be “acceptable” in the sense of being compatible with the other ingredients of the formulation and not deleterious to the recipient thereof.

In a further embodiment of the present invention is provided the use of a bivalent GST inhibitor or a pharmaceutically acceptable salt or prodrug form in the manufacture of a medicament for the treatment of cancer; drug-resistant cancer; immunosuppression; immunosuppression during chemotherapy or radiotherapy; myelodysplasia; bone marrow transplantation; malaria; drug-resistant malaria; any of the disorders and conditions as disclosed in U.S. Pat. Nos. 5,763,570, 5,767,086, and 5,955,432; or related disorders involving GST in a mammal including human.

In an alternative aspect of the present invention is provided a method for the treatment of cancer; drug-resistant cancer; immunosuppression; immunosuppression during chemotherapy or radiotherapy; myelodysplasia; bone marrow transplantation; malaria; drug-resistant malaria; any of the disorders and conditions as disclosed in U.S. Pat. Nos. 5,763,570, 5,767,086, and 5,955,432; or related disorders involving GST for the treatment of a mammal, including human comprising the administration of an effective amount of a bivalent GST inhibitor. It will be appreciated by people skilled in the art that treatment extends to prophylaxis as well to the treatment of such diseases.

The bivalent GST inhibitors of the present invention are useful in combinations, formulations and methods for the treatment and prophylaxis of cancer; drug-resistant cancer; immunosuppression; immunosuppression during chemotherapy or radiotherapy; myelodysplasia; malaria; drug-resistant malaria; any of the disorders and conditions as disclosed in U.S. Pat. Nos. 5,763,570, 5,767,086, and 5,955,432; or related disorders involving GST.

The term “combinations” as used herein, includes a single dosage form containing at least one bivalent GST inhibitor of this invention and at least one other pharmaceutical agent, a multiple dosage form, wherein the bivalent GST inhibitor and the pharmaceutical agent are administered separately, but concurrently, or a multiple dosage form wherein the two components are administered separately, but sequentially. In sequential administration, the bivalent GST inhibitor may be given to the patient during the time period ranging from about 5 hours prior to about 5 hours after administration of the pharmaceutical agent. Preferably, the bivalent GST inhibitor is administered to the patient during the period ranging from 2 hours prior to 2 hours following administration of the pharmaceutical agent.

Examples of pharmaceutical agents useful in combination with bivalent GST inhibitors of the present invention include, but are not limited to, melphan, doxorubicin, adriamycin, chlorambucil, cyclophosphamide, carboplatin, bleomycin, cisplatin, GM-CSF, G-CSF, cytokines, amifostine, and chloroquine. In these combinations, the bivalent GST inhibitor and the pharmaceutical agent work in a complementary fashion. The use of the bivalent GST inhibitor in the formulations of this invention advantageously allows the administration of the pharmaceutical agent in dosages previously considered too low to result in therapeutic effects if given alone, or the restoration of efficacy of the pharmaceutical agent in cases where the patient has become resistant to this agent when given alone.

Various dosage forms may be employed to administer the formulations and combinations of this invention. These include, but are not limited to, parenteral administration, oral administration and topical application. The formulations and combinations of this invention may be administered to the patient in any pharmaceutically acceptable dosage form, including those which may be administered to a patient intravenously as bolus or by continued infusion, intramuscularly—including paravertebrally and periarticularly—subcutaneously, intracutaneously, intra-articularly, intrasynovially, intrathecally, intra-lesionally, periostally or by oral, nasal, or topical routes. Such compositions and combinations are preferably adapted for topical, nasal, oral and parenteral administration, but, most preferably, are formulated for oral administration.

Parenteral compositions are most preferably administered intravenously either in a bolus form or as a constant infusion. For parenteral administration, fluid unit dose forms are prepared which contain the bivalent GST inhibitors of the present invention and a sterile vehicle. The bivalent GST inhibitors of this invention may be either suspended or dissolved, depending on the nature of the vehicle and the nature of the particular bivalent GST inhibitors of this invention. Parenteral compositions are normally prepared by dissolving the bivalent GST inhibitors of this invention in a vehicle, optionally together with other components, and filter sterilizing before filling into a suitable vial or ampoule and sealing. Preferably, adjuvants such as a local anesthetic, preservatives and buffering agents are also dissolved in the vehicle. The composition may then be frozen and lyophilized to enhance stability.

Parenteral suspensions are prepared in substantially the same manner, except that the active bivalent GST inhibitor is suspended rather than dissolved in the vehicle. Advantageously, a surfactant or wetting agent is included in the composition to facilitate uniform distribution of its components.

Tablets and capsules for oral administration may contain conventional excipients, such as binding agents, fillers, diluents, tableting agents, lubricants, disintegrants, and wetting agents. The tablet may be coated according to methods well known in the art. Suitable fillers which may be employed include cellulose, mannitol, lactose and other similar agents. Suitable disintegrants include, but are not limited to, starch, polyvinylpyrrolidone and starch derivatives, such as sodium starch glycolate. Suitable lubricants include, for example, magnesium stearate. Suitable wetting agents include sodium lauryl sulfate.

Oral liquid preparations may be in the form of aqueous or oily suspensions, solutions, emulsions, syrups or elixirs, or may be presented as a dry product for reconstitution with water or another suitable vehicle before use. Such liquid preparations may contain conventional additives. These include suspending agents, such as sorbitol, syrup, methyl cellulose, gelatin, hydroxyethylcellulose, carboxymethylcellulose, aluminum stearate gel or hydrogenated edible fats, emulsifying agents which include lecithin, sorbitan monooleate, polyethylene glycols, or acacia, non-aqueous vehicles, such as almond oil, fractionated coconut oil, and oily esters, and preservatives, such as methyl or propyl p-hydroxybenzoate or sorbic acid.

Formulations for topical administration may, for example, be in aqueous jelly, oily suspension or emulsified ointment form.

The dosage and dose rate of the bivalent GST inhibitors of this invention will depend on a variety of factors, such as the weight of the patient, the specific pharmaceutical composition used, the object of the treatment, i.e., therapy or prophylaxis, the nature of the disease to be treated, and the judgment of the treating physician.

According to the present invention, a preferred pharmaceutically effective daily dose of the compounds of this invention is between about 1 μg/kg body weight of the patient to be treated (“body weight”) and about 10,000 μg/kg body weight. Most preferably, the therapeutic and prophylactic compositions of the present invention comprise a dosage of between about 10 μg/kg body weight and about 1000 μg/kg body weight of the bivalent GST inhibitors of this invention. Most preferred combinations comprise the same amount of the bivalent GST inhibitors of this invention and between about 10% and about 200% of the conventional dosage range of the pharmaceutical agent. It should also be understood that a daily pharmaceutically effective dose of either the bivalent GST inhibitors of this invention or the pharmaceutical agent present in combinations of the invention, may be less than or greater than the specific ranges cited above.

Once improvement in the patient's condition has occurred, a maintenance dose of a combination or composition of this invention is administered, if necessary. Subsequently, the dosage or the frequency of administration, or both, may be reduced, as a function of the symptoms, to a level at which the improved condition is retained. When the symptoms have been alleviated to the desired level, treatment should cease. Patients may, however, require intermittent treatment upon any recurrence of disease symptoms.

According to an alternate embodiment of this invention, bivalent GST inhibitors may be used in compositions and methods for ex vivo treatment of cells. Such cells include, but are not limited to, cells of any tissue type, including cells from human, mammalian, murine, canine, feline, equine, bovine, porcine, etc. For example, the bivalent GST inhibitors of the invention can be employed to expand or otherwise modulate hematopoietic cells in bone marrow prior to allogeneic or autologous transplants (see Kauvar et al., U.S. Pat. No. 5,955,432). Patients may be treated using ex vivo techniques whereby expansion of relatively undifferentiated cells from the blood stream. When ex vivo administration is employed, either bone marrow or peripheral blood (including cord blood) or both can be directly contacted with the invention compounds or fractions of these materials may be treated so long as the fractions contain suitable target progenitor cells. Preferred target progenitor cells include CD34⁺ cells, GEMM, and BFU-E.

In order that the invention described herein may be more fully understood, the following examples are set forth. It should be understood that these examples are for illustrative purposes only and are not to be construed as limiting this invention in any manner.

### 6. EXAMPLES

### 6.1 Materials and Methods

Materials. Reduced glutathione was purchased from Sigma. The crosslinking agents ethylenediol bis-ethylamine and tetraethyleneglycol bis-amine were purchased from Molecular Biosciences. N-BOC-1,3-diaminopropane was purchased from Fluka. All other small molecule starting materials were purchased from Aldrich. Fmoc(3-aminomethyl)benzoic acid (Fmoc-AMBA) was also obtained from Fluka. 3,5-di-Fmoc-aminomethylbenzoic acid (Fmoc₂-DAMBA) was prepared by published methods (see Liu et al., Synthesis 14:2078, 2001 and Martin et al., OPPI Briefs 27:117, 1995), then protected as the bis-Fmoc carbamate using Fmoc-OSu and aqueous NaHCO₃ and dioxane. The Tentagel SRAM Rink-type resin (90μ, 0.23 mmol/g substitution) used for solid phase synthesis was obtained from Peptides International.

Solid phase synthesis. Fmoc deprotection was accomplished by suspending the resin in a solution of 20% piperidine/DMF solution (2 ml/100 mg resin) for 5 min, then filtration. This process was repeated twice more, then followed by washing with DMF. Phthaloyl protected amino acids were prepared by published methods (see Bodansky et al., The Practice of Peptide Synthesis, Springer-Verlag, Berlin 1984). Phthaloyl protecting group removal was accomplished by suspending the resin in a 1 M ethanolic solution of hydrazine (2 ml/100 mg resin) overnight. The resin was then filtered and washed with EtOH (“ethanol”), and dried under vacuum. Coupling procedures were typically carried out by dissolving the 3 eq. (“equivalents”) of the N-protected amino acid relative to the solid-phase bound amine, HATU (3 eq.), HOBt (3 eq.) and DIPEA (6 eq.) in DMF (2 ml/mmol amino acid). After a pre-activation period of 3-5 min, the carboxyl component solution was then added to the resin, and the coupling was allowed to proceed for 1 h at rt. The resin was filtered and washed with DMF, CH₂Cl₂ and MeOH, then dried under vacuum. The products were liberated from the resin support using 95% TFA/H₂O (1 ml/100 mg resin) for 1 h at rt. The resin was then filtered and washed with additional 95% TFA/H₂O solution. The filtrate was collected, and the solvent removed by rotary evaporation. The resulting oils were then triturated with Et₂O to yield the final cleaved products as solids.

GST isozymes. Recombinant human glutathione S-transferase A1-1 was expressed in E. coli and purified as described previously (see Dietze et al., Biochemistry 35(37):11938, 1996). The enzyme was incubated with 10 mM dithiothreitol to ensure complete reduction of cysteine residues, followed by extensive dialysis to remove the reducing agent. Recombinant human glutathione S-transferase P1-1 was expressed and purified by the same protocol. Plasmodium GST (pGST) was a gift from Dr. Katja Becker (Justus Liebig University of Giessen), and murine GSTP and GSTA were gifts from Dr. Dave Eaton (University of Washington). Except where otherwise stated (i.e. testing of pGST in Example 6.4.5 and testing of murine GSTs in Example 6.4.7), GST isozymes tested were from human.

GST assays. Assays of GST activity and inhibition were performed with chloro-2,4-dinitrobenzene (CDNB) as the electrophilic substrate. Except where otherwise stated, GST concentration was 20 nM. For IC₅₀ determinations, the concentration of GSH and CDNB were at their respective Kₘs for each enzyme: 250 μM GSH for both enzymes, 750 μM CDNB for GST A-1, and 1.5 mM CDNB for GST P1-1. Rates were determined by measuring absorption at 340 nm (λₘₐₓ of glutathione-dinitrobenzene) for 1 minute on a Beckman DU 7400 spectrophotometer. For IC₅₀ determinations, assays were performed at inhibitor concentrations ranging over at least five orders of magnitude (from 100-fold above the IC₅₀ to 100-fold below). Data were fit to a sigmoidal dose-response function using GraphPad Prism to determine the 50% inhibitory concentration.

Mass analysis. Electrospray ionization mass spectra of compounds were obtained with a Fisons VG Quattro II mass spectrometer fitted with a Z-spray ESI source. Purified compounds were analyzed by direct injection into the instrument, and in all cases their predicted and found masses were equivalent.

### 6.2 Preparation of Molecular Linkers and Reference Linkers

### Example 6.2.1

### Preparation of dibromo alkyl esters (Molecular Linkers)

This Example illustrates the preparation of the representative series of molecular linkers:

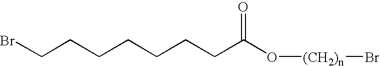

Dibromo alkyl esters were prepared by the acid-catalyzed esterification of 8-bromooctanoic acid with a series of five ω-bromo alkyl alcohols (n=8, 9, 10, 11 and 12). In each of five pre-dried vials, 8-bromooctanoic acid (0.6 mmol, 133.8 mg) was dissolved in freshly distilled toluene. To each of these vials was added para-toluenesulfonic acid (0.05 mmol, 9.5 mg), followed by 0.5 mmol of one of the ω-bromo alcohols (or 2.5 mmol methanol). These solutions were refluxed for one hour with a Dean-Stark trap containing 4 Å molecular sieves to remove the water formed during the esterification. After this time, TLC (“thin-layer chromatography”; methylene chloride development, iodine stain) indicated no remaining 8-bromooctanoic acid (the limiting reagent). The solutions were allowed to cool, then washed sequentially with 5% aqueous sodium bicarbonate, water, and saturated sodium chloride. The toluene was evaporated under a stream of dry air, producing a pale yellow oil. This oil was dissolved in ethyl acetate, and the bicarbonate/water/sodium chloride wash cycle was repeated. The ethyl acetate was dried with anhydrous magnesium sulfate and evaporated under vacuum to yield colorless oil. TLC for each of the six reactions indicated pure products had been isolated.

### Example 6.2.2

### Preparation of the monobromo alkyl ester (Reference Linker)

This Example illustrates the preparation of the representative monofunctional reference linker of the dibromo alkyl ester series:

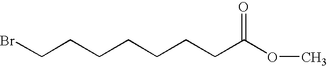

The representative monofunctional reference linker of the dibromo alkyl ester series was prepared by esterification of 8-bromooctanoic acid with methanol according to the procedure of Example 6.2.1.

### Example 6.2.3

### Preparation of bis-ethanolisophthalate and bis-propanolisophthalate (Molecular Linkers)

This Example illustrates the preparation of the representative series of molecular linkers:

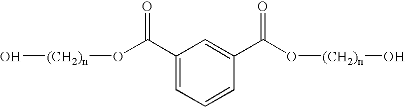

To prepare the isophthalate diols (n=2 and 3), isophthaloyl chloride was esterified to ethylene glycol or 1,3-propanediol. Due to the two equivalent reactive groups of isophthaloyl chloride and the diols, large excesses of the diols were necessary to prevent formation of polymeric species. Ethylene glycol and 1,3-propanediol (4 mL, about 60 mmol) were placed in two separate pre-dried vials. Isophthaloyl chloride (0.5 mmol, 101.5 mg) was placed in each of two separate pre-dried vials and dissolved in 1 mL of dry, freshly distilled THF (“tetrahydrofuran”). Using a dry syringe, the isophthaloyl chloride solution was added dropwise to each diol with vigorous stirring. Stirring was continued for 12 hours, at which time TLC (ethyl acetate development, UV visualization) indicated no remaining isophthaloyl chloride (the limiting reagent). Water (8 mL) was added to each reaction, then the solutions were extracted with 10 mL of toluene. The toluene extract contained a highly non-polar species observed by TLC, but little of the major product. The aqueous solutions were then extracted with 10 mL of either ethyl acetate (ethylene glycol ester) or diethyl ether (1,3-propanediol ester), repeated four times. The five extracts for each product were combined and washed sequentially with water and saturated sodium chloride, then dried with anhydrous magnesium sulfate and evaporated under vacuum. The bis-ethanolisophthalate evaporated to a white solid which was recrystallized from anhydrous diethyl ether; the bis-propanolisophthalate evaporated to a colorless oil.

### Example 6.2.4

### Preparation of bis-propylamineisophthalamide (Molecular Linker)

This Example illustrates the preparation of the representative series of molecular linkers:

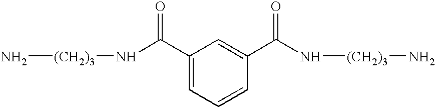

To prepare the bis-propylamineisophthalamide, isophthaloyl chloride (0.1 mmol, 20.3 mg) was placed in a pre-dried vial and dissolved in 3 mL of dry, freshly distilled THF. Neat N-BOC-1,3-diaminopropane (0.286 mmol, 50 μL) was added dropwise from a dry syringe, producing an immediate white precipitate. After stirring for 30 minutes, the precipitate was filtered and the THF solution evaporated under vacuum, leaving an oily residue. This oil was dissolved in 10 mL of ethyl acetate and extracted sequentially with 10 mL each of water, 5% NaHCO₃, water, dilute HCl, and saturated NaCl. The washed ethyl acetate was then dried with anhydrous magnesium sulfate and evaporated under vacuum, leaving a colorless oil. This oil was then dissolved in CHCl₃ (3 mL) and 300 μL trifluoroacetic acid added to effect cleavage of the BOC protecting group and yield the bis-propylamineisophthalamide. After stirring for 30 minutes, TLC (alumina plate, methanol development, UV visualization) indicated complete cleavage had been effected. The solution was then evaporated under vacuum to a colorless oil, which was washed with hexane and dried in vacuo.

### Example 6.2.5

### Preparation of 3,5-bis-[(aminoalkyloylamino)methyl]benzoic acids (Resin-Bound Molecular Linkers)

This Example illustrates the preparation of the representative series of resin-bound molecular linkers:

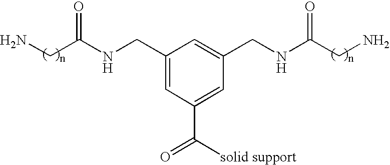

To prepare the 3,5-bis-[(aminoalkyloylamino)methyl]benzoic acid series (n=1, 2, 3, 5 and 7), Fmoc₂-DAMBA was loaded onto the solid phase resin using the HATU/HOBt activation protocol as described in the material and methods section. The Fmoc groups were then removed by treatment with 20% piperidine/DMF, and the resulting free amines were then coupled to phthaloyl protected carboxyl components. The phthaloyl protecting groups were then removed by treatment with hydrazine, to provide the resin-bound 3,5-bis-[(aminoalkyloylamino)methyl]benzoic acid molecular linker.

### Example 6.2.6

### Preparation of 3-[(4-aminobutyrylamino)methyl]benzoic acid (Resin-Bound Reference Linker)

This Example illustrates the preparation of the representative resin-bound monofunctional reference linker of the 3,5-bis-[(aminoalkyloylamino)methyl]benzoic acid series:

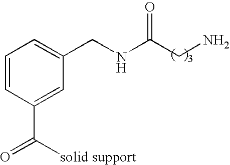

The representative monofunctional reference linker 3-[(4-aminobutyrylamino)methyl]benzoic acid was prepared according to the procedure of Example 6.2.5., except that Fmoc-AMBA was used instead of Fmoc₂-DAMBA for the initial resin loading step.

### Example 6.2.7

### Preparation of bis-GABA3 (Resin-Bound Molecular Linker)

This Example illustrates the preparation of the representative bis-GABA3 (“GABA” is gamma-aminobutyric acid) resin-bound molecular linker:

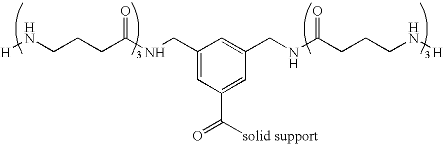

To prepare the resin-bound bis-GABA3 molecular linker, Fmoc₂-DAMBA was loaded onto the solid phase resin using the HATU/HOBt activation protocol as described in the material and methods section. The Fmoc groups were removed by treatment with 20% piperidine/DMF, then Phthaloyl-GABA was coupled to the free amines. The phthaloyl protecting groups were then removed using hydrazine. Fmoc-GABA was then coupled, and the Fmoc groups removed by treatment with 20% piperidine/DMF. This Fmoc-GABA coupling/deprotection step was repeated once more to provide the resin-bound bis-GABA3 molecular linker.

### 6.3 Preparation of Bivalent and Monovalent Inhibitors

### Example 6.3.1

### Bivalent and Monovalent GST Inhibitors Comprising Ligand Domains Derived from GSH (Compounds in Table I)

To prepare the bivalent GSH derivatives of the molecular linkers of Example 6.2.1 (dibromo alkyl esters) and the monovalent GSH derivative of the reference linker of Example 6.2.2 (monobromo alkyl ester), a 1 M reduced glutathione solution was prepared by dissolving 6 mmol GSH (1.842 g) in 4.0 mL water plus 2.0 mL 6 M NaOH (12 mmol NaOH). Aliquots of this solution (0.75 mL, 0.75 mmol GSH) were placed in each of six vials, then 95% ethanol added until a slight cloudiness appeared (total volume about 4 mL). Each ester (0.25 mmol) was placed in a separate vial and dissolved in 0.5 mL of 95% ethanol, then added dropwise to the glutathione solution. These reactions were allowed to stir for 24 hours, and then evaporated under a stream of dry air to a volume of about 1 mL of predominately aqueous solution. This volume was increased to about 4 mL by addition of water, then placed in an ice bath and acidified to pH 3, in each case producing a white precipitate. These precipitates were filtered and washed sequentially with water, acetonitrile, and ether. The washed precipitates were then dried in vacuo.

To prepare the bivalent GSH derivative of the n=3 molecular linker of Example 6.2.5 (3,5-bis-[(aminoalkyloylamino)methyl]benzoic acid) and the monovalent GSH derivative of the reference linker of Example 6.2.6, the free amines of the resin-bound linkers were coupled to bromoacetic acid using the HATU/HOBt activation protocol as described in the Materials and Methods section. The resins were then suspended in a solution containing GSH (5 eq. relative to resin-bound amine) and DIPEA (10 eq.) in H₂O/EtOH/THF (2:3:2) for 2 h at rt. The resins were then washed with H₂O, then EtOH and dried under vacuum. The GSH derivatives were then cleaved from the resin using 95% TFA/H₂O.

### Example 6.3.2

### Bivalent and Monovalent Inhibitors Comprising Ligand Domains Derived from a GSH-NB Conjugate (Compounds in Table II)

To prepare the bivalent derivatives of a GSH-NB conjugate (i.e. the 4-(S-glutathione)-3-nitro-benzoyl conjugate), the molecular linkers of Example 6.2.3 as well as the commercially available diol, tetraethyleneglycol, were esterified with two equivalents of CNBC (4-chloro-3-nitro-benzoyl chloride). Specifically, each of the diols plus ethanol for the monovalent inhibitor (0.25 mmol) were placed in pre-dried vials and dissolved in 1 mL of dry, freshly distilled pyridine. Four portions of CNBC (1.5 mmol) were placed in separate pre-dried vessels and dissolved in 1 mL of dry pyridine. The diol (or ethanol) solutions were added dropwise to the CNBC solutions with stirring, producing an exothermic reaction and an immediate precipitate of pyridine hydrochloride. After 30 minutes, TLC (ethyl acetate development, UV visualization and iodine staining) indicated no remaining diols (the limiting reagents) in any of the reaction media. The precipitate was filtered off of each of the solutions, producing pale yellow solutions in each case. Icewater (10 mL) was added to each solution, resulting in immediate clouding of the solutions. Centrifugation resulted in the formation of off-white solid pellets in the case of the ethyl ester (i.e. reference linker) and bis-ethanolisophthalate diester; the other reactions (tetraethyleneglycol diester and bis-propanolisophthalate diester) yielded pale yellow oils. The solid products were filtered, washed with water, and dried in vacuo. Addition of 95% ethanol to the tetraethyleneglycol diester oil resulted in the formation of a nearly white precipitate which was filtered, washed sequentially with water and ethanol, and dried in vacuo. However, the bis-propanolisophthalate diester remained oily after washing with water and 95% ethanol.

The final step in the preparation of this series of compounds was glutathione substitution of the aryl chlorides. The monovalent precursor, CNB ethyl ester (0.5 mmol, 115 mg), was dissolved in 2 mL of ethanol. GSH (1 mmol, 307 mg) was dissolved in 2 mL water plus 334 μL 6M NaOH (2 mmol NaOH). To this solution 95% ethanol was added until a slight cloudiness appeared (total volume about 6 mL). The CNB ethyl ester solution was then added dropwise to the GSH solution and allowed to stir for 48 hours, producing a yellow solution.

Each of the bivalent precursors, that is, the bis-CNB diesters of the molecular linkers (0.075 mmol) were dissolved in 1 mL acetonitrile. In three separate vials, GSH (0.375 mmol) was dissolved in 1 mL water plus 125 μL 6M NaOH (0.75 mmol NaOH), then acetonitrile (2 mL) was added to this solution. The bis-CNB diester solutions were then each added to one of the GSH solutions. These mixtures were stirred for 48 hours, producing yellow solutions in each case. All four of the above reaction mixtures were then evaporated under a stream of dry air to a volume of about 1 mL of predominately aqueous solution. These were placed in an ice bath and acidified to pH 3 by dropwise addition of 1 M HCl, producing yellow precipitates. After standing for 30 minutes, these mixtures were centrifuged and the supernatants discarded. The solid precipitates were resuspended in dilute HCl, filtered, and washed sequentially with water, acetonitrile, and diethyl ether, then dried in vacuo.

### Example 6.3.3

### Bivalent and Monovalent Inhibitors Comprising Ligand Domains Derived from a GSH-ethacrynic acid Conjugate (Compounds in Table II)

To prepare the bivalent and monovalent derivatives of a GSH-ethacrynic acid conjugate, the n=3 molecular linker of Example 6.2.5 (3,5-bis-[(aminoakyloylamino)methyl]benzoic acid) and the reference linker of Example 6.2.6 were coupled to ethacrynic acid using the HATU/HOBt activation protocol as described in the Materials and Methods section. The resins were then suspended in a solution containing GSH (5 eq. relative to resin-bound amine) and DIPEA (10 eq.) in H₂O/EtOH/THF (2:3:2) overnight at rt. The resins were then washed with H₂O, then EtOH and dried under vacuum. The GSH derivatives were then cleaved from the resin using 95% TFA/H₂O.

### Example 6.3.4

### Bivalent and Monovalent Inhibitors Comprising Ligand Domains Derived from Uniblue A (Compounds in Table III)

To prepare the bivalent uniblue A derivatives of the molecular linker of Example 6.2.4 (bis-propylamineisophthalamide) and the commercially available diamines ethylenediol bis-ethylamine (EDBEA) and tetraethyleneglycol bis-amine (TEGBA), these diamines were conjugated to the α,β-unsaturated sulfone moiety of the commercially available dye uniblue A. The monovalent uniblue A derivative was prepared by conjugating uniblue A to the reference linker, ethylamine. Specifically, conjugation of the diamines with uniblue A was effected by dissolving each diamine (0.1 mmol) in 1 mL of water, then adding this to a solution of uniblue A (0.3 mmol) in 4 mL of water. The pH of these solutions was found to be about 10.5 for the EDBEA and TEGBA solutions, but about 2.0 for the bis-propylamineisophthalamide. The pH of this solution was raised to 10.5 with saturated Na₂CO₃. The solutions were stirred for 2 days and the bis-uniblue A products were purified from the reaction medium by preparative HPLC (“high-pressure liquid chromatography). The products were the final species to elute from a 10 mm i.d. (“internal diameter”) C₁₈ reversed phase column eluted with 30% acetonitrile/70% water. For each product, the pooled dark blue fractions were evaporated under a stream of air; as the acetonitrile was removed, a dark blue precipitate formed. When little color remained in solution, the precipitate was filtered and dried in vacuo. Conjugation of uniblue A with ethylamine to prepare the monofunctional reference compound was performed similarly to the above procedure, but 0.1 mmol of uniblue A (50 mg) was mixed with 0.1 mmol of ethylamine (6.5 μL of a 70% aqueous solution). HPLC purification was performed as above.

To prepare the uniblue A derivatives of DAMBA alone (see 6.1 Materials and Methods) and of the molecular linkers of Example 6.2.5 (3,5-bis-[(aminoalkyloylamino)methyl]benzoic acid) and Example 6.2.7 (bis-GABA₃); and the reference linker of Example 6.2.6, the resin-bound linkers were suspended in a solution of uniblue A (3 eq. relative to resin-bound amine) and DIPEA (3 eq.) dissolved in DMF over 48 h at rt. The resins were then washed with DMF, CH₂Cl₂, then MeOH and dried under vacuum. The uniblue A derivatives were then cleaved from the solid support using 95% TFA/H₂O.

### Example 6.3.5

### Bivalent and Monovalent Inhibitors Comprising Ligand Domains Derived from ethacrynic acid (Compounds in Table IV)

To prepare the ethacrynic acid derivatives of DAMBA alone (see 6.1 Materials and Methods) and of the molecular linkers of Example 6.2.5 (3,5-bis-[(aminoakyloylamino)methyl]benzoic acid) and Example 6.2.7 (bis-GABA₃); and the monovalent ethacrynic acid derivative of the reference linker of Example 6.2.6, the resin-bound molecular linkers were coupled to ethacrynic acid using the HATU/HOBt activation protocol as described in the Materials and Methods section. The ethacrynic acid derivatives were then cleaved from the solid support using 95% TFA/H₂O.

To prepare the ethacrynic acid derivatives of 1,3-phenylenediamine and 1,4-phenylenediamine, ethacrynic acid (2 equiv.) and the corresponding diamine (1 equiv.) was dissolved in DMF (2 mL/mmol diamine). Diisopropylcarbodiimide (DIPCDI, 2 equiv.) was then added to the solution, followed by DMAP (0.1 equiv). The resulting solution was then stirred overnight at room temperature. The reaction mixture was then concentrated by rotary evaporation and partitioned between EtOAc (20 mL) and H₂O (20 mL). Some insoluble material seen at the solvent interface was filtered away using a Buchner funnel. The aqueous layer was removed, and the organic layer washed with 5% NaHCO₃, H₂O, 0.1 N HCl, H₂O and brine. Upon standing, a fine white precipitate formed in the organic layer in both cases. The precipitate was filtered, washed with ether and dried to yield analytically pure samples of the ethacrynic acid derivatives of 1,3- and 1,4-phenylenediamine.

Bis-1,3-ethacrynyl-phenylenediamine: TLC R_{f} (EtOAc/hexanes, 3:2 w/1% AcOH)=0.57; HPLC Rₜ=34.4 min, 97% purity; ESI-MS [MH⁺]=679.0

Bis-1,4-ethacrynyl-phenylenediamine: TLC R_{f} (EtOAc/hexanes, 3:2 w/1% AcOH)=0.36; HPLC Rₜ=33.6 min, 99% purity; ESI-MS [M+Na⁺]=701.0

### 6.4 Inhibition of GST Isozymes By Bivalent Inhibitors of the Invention

### Example 6.4.1

### GST Inhibition with Inhibitors Comprising Ligand Domains Derived from GSH (Table I)

The IC₅₀ was determined each of the 16 to 20 carbon bis-glutathionyl alkyl esters with GST isozymes A1-1 and P1-1 (compounds Ia-Ie). An IC₅₀ was also determined for the monovalent inhibitor (If). Notably, each of the bis-glutathionyl alkyl esters exhibits an IC₅₀ more than one order of magnitude lower than the monovalent benchmark compound and six orders of magnitude lower than Kₘ of glutathione. From this data, it is evident that the bivalent inhibitors exhibit between 10- and 100-fold greater affinities than the corresponding monovalent inhibitor. The bivalent compound Ic has 2-fold greater affinity for the GSTP1-1 isozyme than the GSTA1-1.

### Example 6.4.2

### GST Inhibition with Inhibitors Comprising Ligand Domains Derived from a GSH Conjugate (Table II)

The IC₅₀ of GST isozymes P1-1 and A1-1 were determined for bivalent compounds IIa-IIc and IIe using the CDNB assay. An IC₅₀ was also determined for the monovalent compound IIf. The IC₅₀ for the GS-EA ligand domain (i.e. the glutathione-ethacrynic acid conjugate) is from the prior art (see Ploemen et al., Xenobiotica 23(8):913). The IC₅₀ of bivalent inhibitor IIc indicates that this compound has approximately 100-fold greater affinity for GSTP1-1 than the GS-NB ligand domain alone. With regard to the flexibility of the molecular linker, the relatively more inflexible bivalent inhibitors IIb and IIc had greater affinity the more flexible bivalent inhibitor IIa. Whereas the GS-NB ligand domain exhibits no substantial isozyme specificity, bivalent inhibitor IIc exhibits a 10-fold greater affinity for GSTP1-1 than for GSTA1-1. Although the ligand spacing of bivalent inhibitors IIa and IIb are equivalent, the affinity of GSTA1-1 is 18-fold greater for IIb than IIa, indicating that the particular composition of the molecular linker enhances the affinity of bivalent inhibitor IIb relative to IIa. Of further significance, a similar change in affinity is not found for the GSTP1-1 isozyme, indicating that the molecular linker also increases the isozyme selectivity of IIb relative to IIa, with the ratios of GSTA1-1 to GSTP1-1 affinities being 0.6 and 6.4 for IIb and IIa, respectively (i.e. an 11-fold difference in these ratios). Using an alternative GSH conjugate, GS-EA, bivalent inhibitor IIe has 30-fold greater affinity for GSTP1-1 than the ligand domain IIg alone. These data further demonstrate that the molecular linker enhances the affinity of these inhibitors, with the monovalent IIf inhibitor having approximately 7-fold greater affinity for GSTP1-1 than the ligand domain IIg.

### Example 6.4.3

### GST Inhibition with Inhibitors Comprising Ligand Domains Derived from Uniblue A (Table III)

The CDNB assay was employed to determine IC₅₀ for bivalent compounds IIIa-IIIc and compounds IIIe-IIIk with GST isozyme P1-1, and for bivalent compounds IIIa-IIIc also with GST isozyme A1-1. An IC₅₀ was also determined for the ligand domain IIId and monovalent compound IIIl.

The highest affinity compound IIIh exhibits an IC₅₀ approximately 220-fold lower than the ligand domain IIId alone. With regard to the flexibility of the molecular linker, both relatively flexible and inflexible molecular linkers yielded high and low affinity inhibitors (e.g. compare IIIa and IIIh). These data together demonstrate that the bivalent uniblue A inhibitors have greater than 200-fold the affinity for GSTP1-1 than the corresponding ligand domains alone, and that this affinity is at least 5000 times more specific for the GSTP1-1 isozyme than the GSTA1-1 isozyme. With an IC₅₀ on the order of 22 nM, the bivalent inhibitor IIIh is the tightest-binding inhibitor of GSTP1-1 of which we are aware.

Of the bivalent uniblue A compounds tested against the GSTA1-1 isozyme, most did not exhibit any inhibition of GSTA1-1 even up to a concentration of 10 μM. However, the affinity of this series could be tailored to bind nearly equally well to the P1-1 and A1-1 isozymes as was found for compound IIIj, which had an IC₅₀ of approximately 240 nM for both isozymes.

The data from this series also demonstrates that the molecular linker enhances the affinity of the bivalent and monovalent uniblue A inhibitors, with the monovalent compound IIIl exhibiting approximately 6-fold greater affinity for GSTP1-1 than the ligand domain IIId.

### Example 6.4.4

### GST Inhibition with Inhibitors Comprising Ligand Domains Derived from ethacrynic acid (Table IV)

The IC₅₀ of GST isozymes P1-1 and A1-1 were determined for bivalent compounds IVa-IVi using the CDNB assay. An IC₅₀ was also determined for the monovalent compound IVj. The IC₅₀ for ethacrynic acid (IVk) is from the prior art (see Ploemen et al., Xenobiotica 23(8):913). This series contained high-affinity compounds for both the P1-1 and A1-1 isozymes, wherein the affinity was strongly dependent on the particular distance separating the ligand domains. For the A1-1 isozyme, the highest affinity compound IVe (IC₅₀=12 nM) exhibits an IC₅₀ approximately 406-fold lower than the ligand domain IVk alone, and 1100-fold lower than the monovalent inhibitor IVj. For the P1-1 isozyme, the highest affinity compound IVh (IC₅₀=31 nM) exhibits an IC₅₀ approximately 129-fold lower than the ligand domain IVk alone, and 403-fold lower than the monovalent inhibitor IVj. These data together demonstrate that the bivalent ethacrynic acid inhibitors have greater than 100-fold and 400-fold the affinity for GSTP1-1 and GSTA1-1 than the corresponding ligand domains alone, respectively.

Whereas the ligand domain IVk exhibits no substantial isozyme specificity, the bivalent inhibitor IVe exhibits a 97-fold greater affinity for GSTA1-1 than for GSTP1-1. Alternatively, the affinity of this series can be tailored to bind nearly equally well to the P1-1 and A1-1 isozymes as was found for compound IVg, which had an IC₅₀ of approximately 40 nM for both isozymes.

For two of the inhibitors tested (IVc\hat{ } and IVg\hat{ }), it was found that removal of the central amide moiety had no substantial effect on their inhibitory characteristics when compared to the corresponding inhibitors that had the amide moiety (IVc and IVg). These results suggest that the central amide moiety is not significantly involved in mediating binding of the inhibitor to either of the isozymes.

Since EA is known to be enzymatically conjugated to GSH by GSTs, these bivalent inhibitors might be predicted to be potential substrates for GSTA1-1 or GSTP1-1, such that they would also be catalytically conjugated with GSH. However, using an assay that measured absorbance changes at a wavelength of 270 nm, testing of IVg* yielded no GSH conjugation with either GSTA1-1 or GSTP1-1. In contrast, EA was readily conjugated to GSH under the same conditions.

### Example 6.4.5

### Plasmodium GST (PGST) Inhibition with Inhibitors Comprising Ligand Domains Derived from Uniblue A and ethacrynic acid

The IC₅₀ values for pGST were determined for the bivalent compounds from Tables III and IV using the CDNB assay. The most potent inhibitors were compounds IVg, IVg\hat{ } and IVi from Table IV, with IC₅₀ values of approximately 3300, 1500 and 5500 nM, respectively.

### Example 6.4.6

### In Vivo Inhibition of Plasmodium

A single dose of compound IVg\hat{ } was added to ring stages of chloroquine resistant Plasmodium falciparum strain Dd2 and incubated for 48 hours. The compound inhibited parasite growth with an EC₅₀ of 412 nM. An isobologram analysis was performed in two different chloroquine resistant parasites comparing IVg\hat{ } with chloroquine (AN143 with a PNG/South America genotype and INDO21 with the Indonesia genotype). No evidence of synergy with chloroquine was observed.

### Example 6.4.7

### Murine GST Inhibition with Inhibitors Comprising Ligand Domains Derived from ethacrynic acid

Using the CDNB assay, the IC₅₀ values for murine GSTP were determined for the bivalent compounds IVc\hat{ } and IVg\hat{ } to be 990 nM and 18 nM, respectively. These data together demonstrate that the bivalent ethacrynic acid inhibitors performed similarly with the murine GSTP isozyme as they did with the corresponding human isozyme.

### Example 6.4.8

### Inhibitor Treatment of Genetically Modified Human 293 Cells

The purpose of this example is to demonstrate the in vivo ability of a bivalent inhibitor to block the protective effect of GST P1-1 overexpression on cell killing by photodynamic therapy, and to established the in vivo IC₅₀ in a cell line of a bivalent inhibitor alone. Cells used in the assay were human kidney 293 fibroblasts transfected with the IRES vector plus and minus the coding region for human GST P1-1. Cells were cultured in DMEM supplemented with 10% FBS and 1% penicillin-streptomycin and maintained at 37° C. and 5% CO₂.

Once the cells reached 80% confluency, the original media was removed and replaced with either (a) fresh media alone; (b) fresh media containing 1 uM hypericin (a porphyrin analogue employed in photodynamic cancer therapy), (c) the desired concentration of GST inhibitor IVg\hat{ }, and (d) 1 μM hypericin plus the inhibitor 1 μM IVg\hat{ }. The cells were incubated for a further 24 hours, in the dark, to allow the hypericin to be incorporated. The media from each plate was then removed and placed into a corresponding 15 ml centrifuge tube. This media is saved to monitor the “floater” cells that may have sloughed off during manipulations and treatment. The attached cells were washed two times with 1×PBS and fresh media is added back to the plates. The plates which were incubated overnight in the presence of hypericin were overlayed with DMEM without the phenol red indicator dye or 1×PBS supplemented with 10% FBS.

All reactions were performed in triplicate and all plates were maintained in the dark at 37° C. until light treatment or harvest. Replicates included untreated cells and cells incubated with hypericin but not exposed to light. For light treatment, cultured cells in the absence and presence of the photosensitizer, hypericin, were exposed for 5 minutes to a xenon arc lamp with an inline 590 nm long pass filter. Cells exposed to light in the absence of hypericin served to monitor the effects of long wave light and heat generated by the arc lamp.

Following light treatment, cells were trypsinized, removed to their corresponding 15 ml tubes and maintained on ice until analyzed via flow cytometry. Fluorophores used to monitor the state of the cells included Hoechst 33258 and 33342, monochlorobimame (for glutathione levels) and Annexin V 350 for apoptotic state.

The results are shown in Table V. GST P1-1 overexpressing cells are, on average, 46% less damaged than IRES (i.e. no GST P1-1 overexpression) control cells post hypericin and light treatment in the absence of GST inhibitor IVg\hat{ }. The GST P1-1 cells are damaged to a similar extent as the control cells post hypericin and light treatment in the presence of 1 μM of the GST inhibitor. The IC₅₀ of inhibitor IVg\hat{ } for these cells was found to be 1.13 μM+/−0.2 SD.

TABLE I
GST inhibitors comprising ligand domains derived from GSH

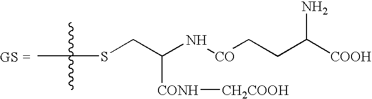

| Compound | Structure |
|---|---|
| Ia | 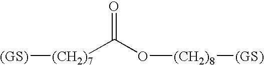 |
| Ib | 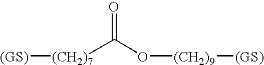 |
| Ic | 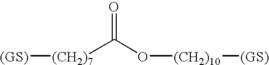 |
| Id | 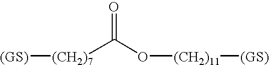 |
| Ie | 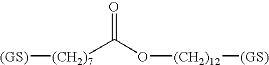 |
| If* | 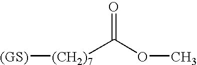 |
| Ig | 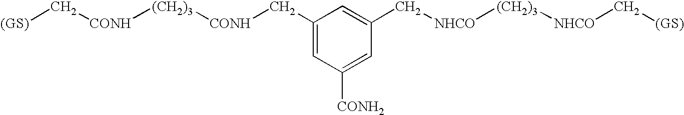 |
| Ih* | 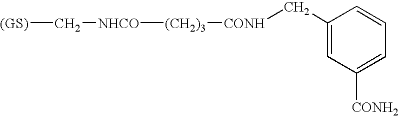 |
| Ii* | (GS)H |

| | | | Approximate |
|---|---|---|---|
| | | IC₅₀ | Distance |
| | | GSTP1-1/ | Separating |
| | | GSTA1-1 | Ligand |
| Compound | Type | (nM) | Domains (Å) |
| Ia | Bivalent | 360/200 | 23.3 |
| Ib | Bivalent | 300/340 | 24.5 |
| Ic | Bivalent | 80/160 | 25.7 |
| Id | Bivalent | 200/110 | 26.9 |
| Ie | Bivalent | 190/140 | 28.3 |
| If* | Monovalent | 3800/3800 | Not applicable |
| Ig | Bivalent | 24800/>10000 | 28.3 |
| Ih* | Monovalent | 39300/>10000 | Not applicable |
| Ii* | Ligand Domain | Kₘ = 250 million | Not applicable |

*Not a bivalent inhibitor of the subject invention. Abbreviations: GS = glutathione ligand domain; and (GS)H = glutathione.

TABLE II
GST inhibitors comprising ligand domains derived from GSH conjugates

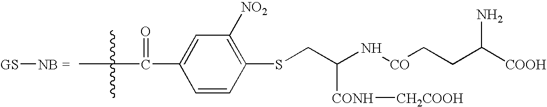

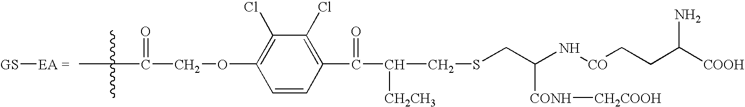

| Compound | Structure |
|---|---|
| IIa | 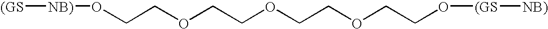 |
| IIb | 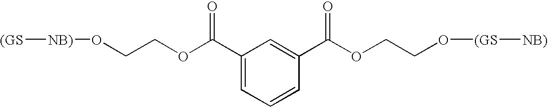 |
| IIc | 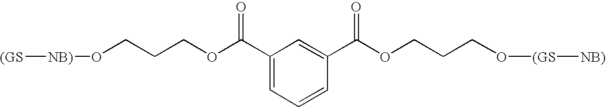 |
| IId* | GS—NB—O—CH₂CH₃ |
| IIe | 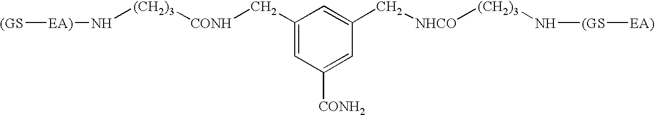 |
| IIf* | 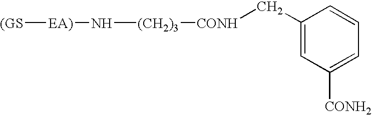 |
| IIg* | (GS—EA)—OH |

| | | | Approximate |
|---|---|---|---|
| | | IC₅₀ | Distance |
| | | GSTP1-1/ | Separating |
| | | GSTA1-1 | Ligand |
| Compound | Type | (nM) | Domains (Å) |
| IIa | Bivalent | 4500/29000 | 18.1 |
| IIb | Bivalent | 2900/1600 | 18.1 |
| IIc | Bivalent | 280/3300 | 20.6 |
| IId* | Ligand domain | 21000/30000 | Not applicable |
| IIe | Bivalent | 353/3.8 | 23.3 |
| IIf* | Monovalent | 1500/5833 | Not applicable |
| IIg* | Ligand domain | 11000/− | Not applicable |

*Not a bivalent inhibitor of the subject invention. Abbreviations: GS—NB = the 4-(S-glutathione)-3-nitro-benzoyl conjugate ligand domain; GS—EA = the glutathione-ethacrynic acid conjugate ligand domain; and (GS—EA)—OH = glutathione-ethacrynic acid conjugate.

TABLE III
GST inhibitors comprising ligand domains derived from Uniblue A

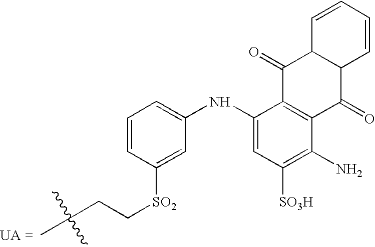

| | | | | Approximate |
|---|---|---|---|---|
| | | | IC₅₀ | Distance |
| | | | GSTP1-1/ | Separating |
| Com- | | | GSTA1-1 | Ligand |
| pound | Structure | Type | (nM) | Domains (Å) |
| IIIa | 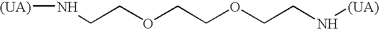 | Bivalent | 44/>100000 | 14.2 |
| IIIb | 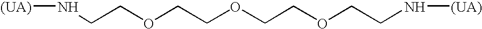 | Bivalent | 72/>100000 | 18.1 |
| IIIc | 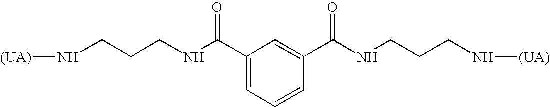 | Bivalent | 440/>100000 | 20.6 |
| IIId* | (UA)—NH—CH₂CH₃ | Ligand Domain | 5000/>100000 | Not applicable |
| IIIe | 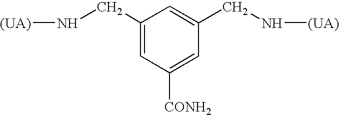 | Bivalent | 557/>10,000 | 10.3 |
| IIIf | 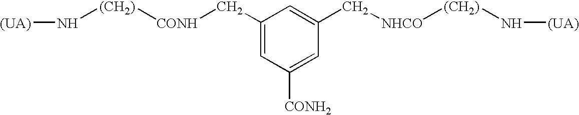 | Bivalent | 437/>1000 | 18.1 |
| IIIg | 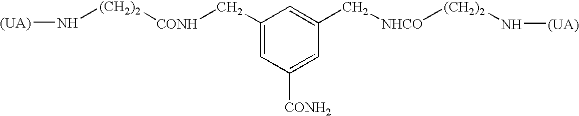 | Bivalent | 162/2500 | 20.6 |
| IIIh | 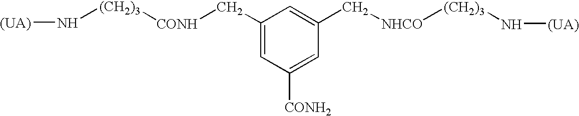 | Bivalent | 22/>1000 | 23.2 |
| IIIi | 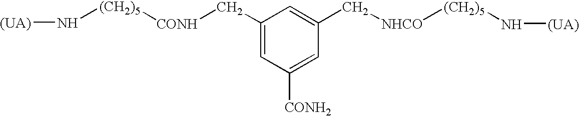 | Bivalent | 203/>1000 | 28.4 |
| IIIj | 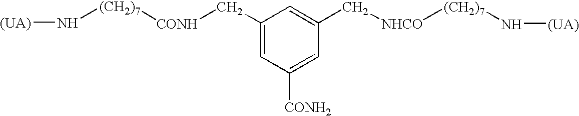 | Bivalent | 267/>220 | 33.5 |
| IIIk | 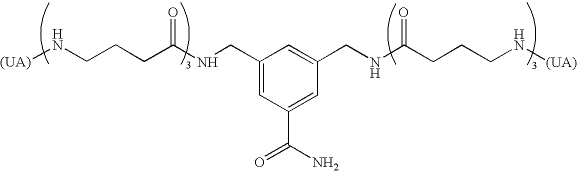 | Bivalent | 600/none | 46.4 |
| IIIl* | 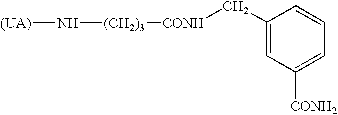 | Monovalent | 854/>20000 | Not applicable |

*Not a bivalent inhibitor of the subject invention. Abbreviations: UA = Uniblue A ligand domain.

TABLE IV
GST inhibitors comprising ligand domains derived from ethacrynic acid

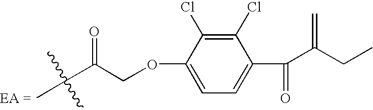

| | | | | Approximate |
|---|---|---|---|---|
| | | | IC₅₀ | Distance |
| | | | GSTP1-1/ | Separating |
| Com- | | | GSTA1-1 | Ligand |
| pound | Structure | Type | (nM) | Domains (Å) |
| IVa | 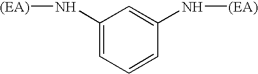 | Bivalent | >10,000/741 | 7.8 |
| IVb | 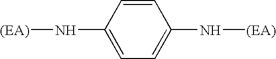 | Bivalent | >10,000/1307 | 9.1 |
| IVc | 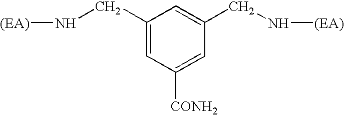 | Bivalcnt | 992.4/29.7 | 10.4 |
| IVc\hat{ } | 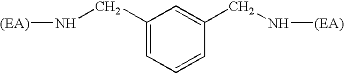 | Bivalent | 995/25 | 10.4 |
| IVd | 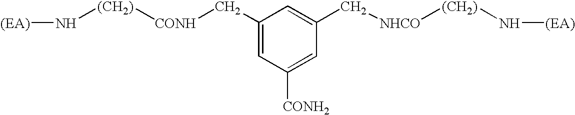 | Bivalent | 633.7/23.0 | 18.1 |
| IVe | 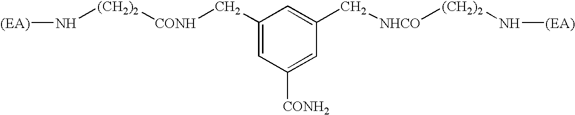 | Bivalent | 1199/12.3 | 20.6 |
| IVf | 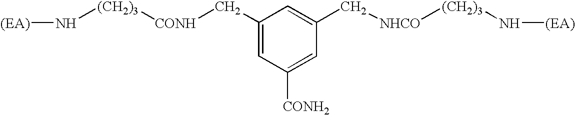 | Bivalent | 589/14 | 23.2 |
| IVg | 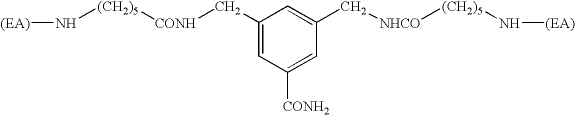 | Bivalent | 46.1/40.2 | 28.4 |
| IVg\hat{ } | 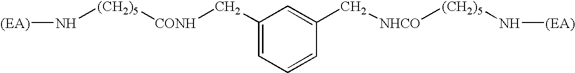 | Bivalent | 27/64 | 28.4 |
| IVh | 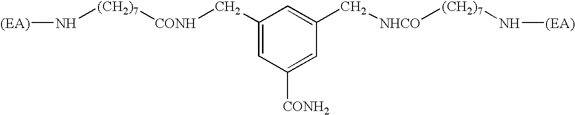 | Bivalent | 31.0/54.0 | 33.5 |
| IVi | 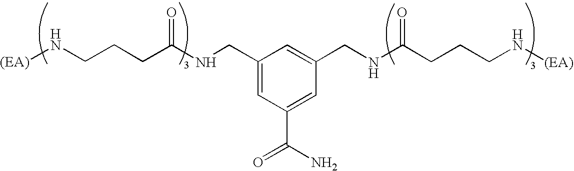 | Bivalent | 142/96.5 | 46.4 |
| IVj* | 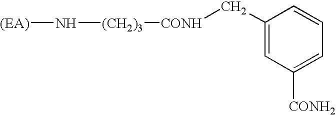 | Monovalent | 12500/13580 | Not applicable |
| IVk* | (EA)—OH | Ligand | 4000/5000 | Not applicable |

*Not a bivalent inhibitor of the subject invention. Abbreviations: EA = ethacrynic acid ligand domain; and (EA)—OH = ethacrynic acid.

TABLE V
Effect of DYM-VII-073 on genetically modified cells

| | Mean % Cell Death | SD | N |
|---|---|---|---|
| (1) IRES control (no inhib) | 3.40 | 0.10 | 3.00 |
| (2) GSTP control (no inhib) | 4.03 | 0.98 | 3.00 |
| (3) IRES + hypericin/hv | 24.63 | 2.46 | 3.00 |
| (4) GSTP + hypericin/hv | 11.27 | 2.35 | 3.00 |
| (5) IRES + inhibitor/hypericin/hv | 20.27 | 1.76 | 3.00 |
| (6) GSTP + inhibitor/hypericin/hv | 22.53 | 2.61 | 3.00 |

Abbreviations: IRES, cells transfected with vector containing no GST coding sequences; GSTP, cells transfected with vector containing the GSTP1-1 coding sequence such that they overexpress GSTP1-1; and hv, 590 nm light treatment.

The present invention is not to be limited in scope by the specific embodiments disclosed in the examples which are intended as illustrations of a number of aspects of the invention and any embodiments which are functionally equivalent are within the scope of this invention. Indeed, various modifications of the invention in addition to those shown and described herein will become apparent to those skilled in the art and are intended to fall within appended claims.
A number of references have been cited, the entire disclosures of which are incorporated herein by reference.

1. A bivalent inhibitor having affinity for a dimeric GST isozyme, wherein the bivalent inhibitor comprises two ligand domains covalently bonded to one another by a molecular linker, wherein the ligand domains have affinity for one or more monomers in the dimeric GST isozyme and are separated from one another by a distance ranging from about 5 to about 100 Å.

2. The bivalent inhibitor of claim 1, wherein the bivalent inhibitor is represented by the formula:
D¹-L-D²
or a pharmaceutically acceptable salt thereof, where D¹ and D² are the two ligand domains and L is the molecular linker, wherein the ligand domains D¹ and D² are independently selected from the groups a) and b) consisting of:
a) amides; acids; or alkyl (1-10C), aryl (1-10C) or aralkyl (7-12C) esters of a monovalent radical of the formula: 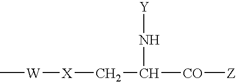
wherein W is selected from the group consisting of a direct link or a mono- or disubstituted or unsubstituted hydrocarbon radical (1-20C) optionally containing 1 or 2 nonadjacent heteroatoms (O, S, or N), and wherein said substitution is selected from the group consisting of halo, —NO, —NO₂, —NR₂, —OR, and —SR, wherein R is H or lower alkyl (1-4C);
wherein X is selected from the group consisting of S, O, CH₂ and direct link;
wherein Y is selected from the group consisting of 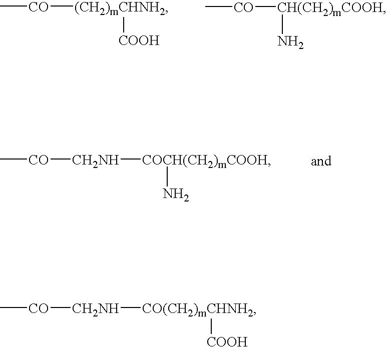
wherein m is 1 or 2;
wherein Z is selected from the group consisting of glycine, valine, alanine, β-alanine, 4-aminobutyric acid, aspartic acid, phenyl glycine, histidine, tryptophan, tyrosine, and phenylalanine, wherein the phenyl group of phenylalanine or phenyl glycine may optionally contain a single substitution selected from the group consisting of halo, OR, and SR, wherein R is H or alkyl (1-4C) linked through a peptide bond to the remainder of the compound;
b) amides; acids; or alkyl (1-10C), aryl (1-10C) or aralkyl (7-12C) esters of a monovalent radical selected from the group consisting of 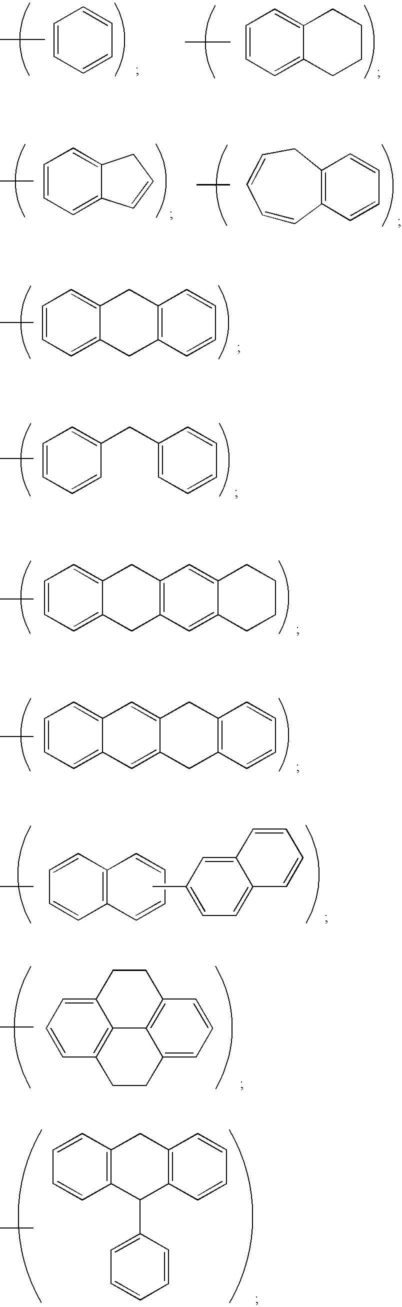 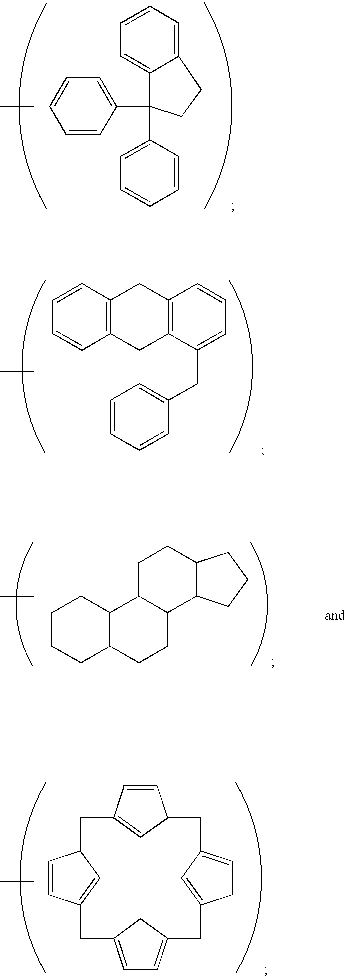
wherein the monovalent radical may optionally be interrupted by at least one heteroatom selected from the group consisting of O, N and S;
wherein the monovalent radical may optionally contain one or more substitutions selected from the group consisting of Cl, Br, F, I, —NH₂, —OH, ═O, —NO₂, —COOH, —CHO, —CO(alkyl), —CO(aryl), —SO₃H, —SO₂NH₂, —SO₂(alkyl), —SO₂(aryl), —CF₃, alkyl, alkoxyalkyl, aryl, and aralkyl;
the molecular linker L is selected from the group consisting of 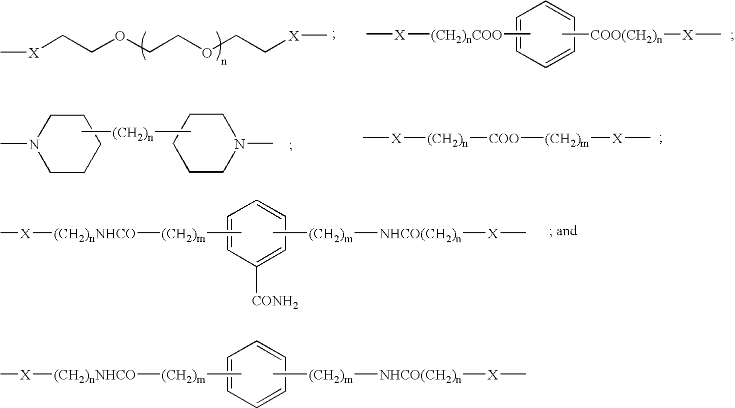
wherein X is N, O, S, CH(AA)NH[COCH(AA)NH]ₖ, direct link, N-alkyl, N-aryl, CON-alkyl, or CO-alkyl; wherein AA is a natural or unnatural amino acid side chain;
wherein k is 0-8, m is 0-8, n is 0-8; and
whereby the bivalent inhibitor to the dimeric GST isozyme has diverse properties.

3. The bivalent inhibitor of claim 1, wherein the ligand domains are separated by a distance ranging from about 5 to about 25 Å.

4. The bivalent inhibitor of claim 1, wherein the molecular linker separates the ligand domains with about 5 to about 100 molecular bonds.

5. The bivalent inhibitor of claim 1, wherein the affinity between the bivalent inhibitor and the dimeric GST isozyme is characterized by a dissociation constant less than 100 nM.

6. The bivalent inhibitor of claim 1, wherein the affinity between the bivalent inhibitor and the dimeric GST isozyme is at least 10-fold greater than the affinity of each of the ligand domains for the dimeric GST isozyme.

7. The bivalent inhibitor of claim 1, wherein the molecular linker enhances the affinity of the bivalent inhibitor.

8. The bivalent inhibitor of claim 1, wherein the molecular linker increases isozyme selectivity of the bivalent inhibitor.

9. The bivalent inhibitor of claim 1, wherein the bivalent inhibitor is a component in a composition comprising a pharmaceutically acceptable carrier.

10. The bivalent inhibitor of claim 1, in which the affinity is for substantially one GST isozyme.

11. The bivalent inhibitor of claim 10, wherein the GST isozyme is the GSTP1-1 isozyme.

12. The bivalent inhibitor of claim 1, in which the affinity is for substantially one GST class.

13. The bivalent inhibitor of claim 12, wherein the GST class is the π class of GST isozymes.

14. The bivalent inhibitor of claim 12, in which the affinity for one GST class is 10-fold greater than the affinity for another GST class.

15. The bivalent inhibitor of claim 1, wherein both ligand domains are the same structure.

16. The bivalent inhibitor of claim 1, wherein the ligand domains are different structures.

17. The bivalent inhibitor of claim 1, wherein the ligand domains are independently selected from the group consisting of glutathione, glutathione analogues, glutathione conjugates, ethacrynic acid, cibacron blue, uniblue A, doxorubicin, gossypol, hematin, rose bengal, sulfobromophthalein, indomethacin, piriprost, eosin b, eosin y, a synthetic or naturally occurring drug; a peptide, a small organic molecule; and mixtures thereof.

18. The bivalent inhibitor of claim 1, wherein the molecular linker comprises a polynucleotide; a peptide; a saccharide; a cyclodextrin; a dextran; polyethylene glycol; polypropylene glycol; polyvinyl alcohol; a hydrocarbon; a polyacrylate; an alkyl chain interrupted by one or more atoms of O, S, or N atom, carbonyl, amide or aromatic group; an amino-, hydroxy-, thio- or carboxy-functionalized silicone; or a combination thereof.

19. The bivalent inhibitor of claim 1 is derivatized with a toxin or chemotherapeutic.

20. A method for inhibiting a dimeric GST isozyme in a cell, comprising preparing a bivalent inhibitor comprising two ligand domains covalently bonded to one another by a molecular linker, wherein the ligand domains have affinity for one or more monomers in the dimeric GST isozyme and are separated from one another by a distance ranging from about 5 to about 100 Å, and introducing said bivalent inhibitor to the cell.

21. The method of claim 20, in which the cell comprises one GST isozyme.

22. The method of claim 20, in which the cell comprises two GST isozymes.

23. The method of claim 20, comprising the additional step of removing the cell from a patient, and introducing the bivalent inhibitor to the cell outside of the patient.

24. The method of claim 20, in which the cell is a bone marrow cell.

25. A method for inhibiting a dimeric GST isozyme in a patient comprising preparing a bivalent inhibitor comprising two ligand domains covalently bonded to one another by a molecular linker, wherein the ligand domains have affinity for one or more monomers in the dimeric GST isozyme and are separated from one another by a distance ranging from about 5 to about 100 Å, and administering to the patient a therapeutically effective amount of the bivalent inhibitor or a pharmaceutically acceptable salt thereof.

26. The method of claim 25, wherein said bivalent inhibitor is administered by oral, transdermal or parenteral means.

27. The method of claim 25, wherein said bivalent inhibitor is administered in an amount of from about 0.01 to about 10 mg/kg per dose.

28. The method of claim 25, in which substantially one GST isozyme is inhibited.

29. The method of claim 25, in which substantially one GST class is inhibited.

30. The method of claim 25, in which greater than one GST isozyme is inhibited.

31. The method of claim 25, in which the GST isozymes are from the π class of GST isozymes.

32. The method of claim 25, in which the bivalent inhibitor inhibits the enzyme catalysis of the dimeric GST isozyme.

33. The method of claim 25, in which the bivalent inhibitor inhibits binding of the dimeric GST isozyme by another protein.

34. The method of claim 25, in which the GST isozyme inhibited is in a tumor, an infectious agent, or a bone marrow of the patient.

35. The method of claim 25, wherein the bivalent inhibitor is derivatized with a toxin or chemotherapeutic agent.

36. The method of claim 25, wherein the linker is derivatized to optimize the absorption, distribution, metabolism or excretion of the bivalent inhibitor.

37. The method of claim 25, wherein the bivalent inhibitor is used for the treatment of cancer; drug-resistant cancer; immunosuppression; immunosuppression during chemotherapy or radiotherapy; myelodysplasia; bone marrow transplantation; malaria; and drug-resistant malaria.

38. The method of claim 25, wherein the bivalent inhibitor is used in combination with another pharmaceutical agent.

39. A method for inhibiting a multimeric isozyme, comprising preparing a bivalent inhibitor comprising two ligand domains covalently bonded to one another by a molecular linker, wherein the linker is of a length that permits the bivalent inhibitor to inhibit substantially one of a plurality of multimeric isozymes, and contacting the multimeric isozyme with the bivalent inhibitor.

40. A bivalent inhibitor comprising two ligand domains covalently bonded to one another by a molecular linker, wherein the linker is of a length that permits the bivalent inhibitor to inhibit substantially one of a plurality of multimeric isozymes.
